(19) United States
(12) Patent Application Publication
(10) Pub. No.: US 2025/0255906 A1
(43) Pub. Date: Aug. 14, 2025

(54) FORMULATIONS AND METHODS FOR CELLULAR THERAPIES INVOLVING CHIMERIC ANTIGEN RECEPTORS

(71) Applicant: Mitrix Bio Inc., Pleasanton, CA (US)

(72) Inventor: Thomas B. Benson, Los Altos, CA (US)

(21) Appl. No.: 19/098,583

(22) Filed: Apr. 2, 2025

Related U.S. Application Data
(63) Continuation of application No. PCT/US2023/034376, filed on Oct. 3, 2023.
(60) Provisional application No. 63/378,657, filed on Oct. 6, 2022.

(51) Int. Cl. A61K 35/19; A61K 35/17; A61K 40/31; C12N 5/0783; C12N 5/0786

(52) U.S. Cl. CPC A61K 35/19; A61K 35/17; A61K 40/31; C12N 5/0636; C12N 5/0645; C12N 2502/115

(57) ABSTRACT

Energetics of chimeric antigen receptor (CAR)-containing cells can be enhanced by obtaining platelets from blood, obtaining mitlets from the platelets, and co-incubating the mitlets with the CAR-containing cells. Uptake of the mitlets into the CAR-containing cells yields enhanced CAR-containing cells having increased metabolic activity. The enhanced CAR-containing cells can be for treatment of an indication treatable by the CAR-containing cells. The mitlets can include coated mitochondria or vesicle-enclosed mitochondria.

### CROSS-REFERENCE TO RELATED APPLICATIONS

The present application is a continuation of PCT Patent Application No. PCT/US2023/034376, filed Oct. 3, 2023, which claims the benefit of priority of U.S. Provisional Application No. 63/378,657, filed Oct. 6, 2022, each of which is incorporated by reference in its entirety herein.

### BACKGROUND

The present technology generally relates to formulations and methods for enhancing chimeric antigenic receptors (CAR)-based technologies, and more specifically, to formulations and methods for using mitlets to increase the energetics and persistence of CAR-expressing cells in CAR-based therapies.

The present technology generally relates to formulations and methods for enhancing chimeric antigenic receptors (CAR)-based technologies, and more specifically, to formulations and methods for using mitlets to increase the energetics and persistence of CAR-expressing cells in CAR-based therapies.

Clinical therapies based on cells expressing CAR have shown promise in their clinical outcomes against a variety of cancer indications. CAR are synthetic receptors that are engineered to bind to specific antigens on the surface of targeted cells (e.g., cancer cells) and trigger a cytotoxic response that results in the death of the target cells. Typically, CARs are expressed in immunological cells (e.g., T cells and Natural Killer (NK) cells). CARs include an extracellular domain (e.g., single chain variable fragment, scFv) that recognizes and binds to the antigen of a targeted cell, a transmembrane domain, a spacer domain (e.g., IgG hinge region) that links the extracellular and transmembrane domains, and a co-stimulatory domain.

In a typical CAR-T cell treatment, T cells are removed from the patient. These autologous T cells are sent to a lab where a gene for a chimeric tumor antigen-specific receptor is added to these T cells. This addition leads to the expression of the gene in the T cell, thereby creating a CAR-T cell. The CAR-T cells are expanded to reach sufficient numbers for an effective dose. The lab freezes and ships these expanded CAR-T's back to the hospital or treatment center. At the hospital or treatment center, these frozen CAR-T cells are thawed and injected into the patient. While CAR-related therapies have shown promise against various cancer indications, there remains several issues with this type of therapy that limit its widespread adoption.

Some of the issues related to CAR-based therapies include: (1) onset of cytokine release syndrome (CRS) and neurotoxicity, particularly notable in CD19 CAR-T cell therapy; (2) on-target off-tumor effects that may be related to the recognition of molecular biomarkers that are expressed on healthy tissue (e.g., B cell aplasia in anti-CD19/CD20 CAR-T cell therapy); (3) antigen escape/loss may lead to disease relapse (e.g., CD19-negative relapse in B-cell malignancy; (4) Graft-versus-Host Disease (GVHD); (5) HLA restriction; (6) insufficient sources of T cells; (7) slow, costly growth and expansion; and (8) limited persistence after infusion into the patient. Lu H, et al. “From CAR-T Cells to CAR-NK Cells: A Developing Immunotherapy Method for Hematological Malignancies” Front Oncol. 2021 Aug. 6; 11:720501. The limited persistence may be due to weakening of the CAR-T cells due to the expansion and freeze-thaw process that have degraded the quality of the mitochondria, or the patient was at an age where the immunological cells were already weak. To address these issues, various formulations and methods are provided to incorporate mitlets (mitochondria encased in vesicles; or coated-mitochondria) into CAR-based therapies as well as during the generation and expansion of CAR-expressing cells used in such therapies.

### SUMMARY

The formulations and methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and desirous properties. The components, aspects, and steps may also be arranged and ordered differently. After considering this discussion, and particularly after reading the section entitled “Detailed Description”, one will understand how the features of the devices and methods disclosed herein provide advantages over other known devices and methods.

In some embodiments, a method of enhancing activity of chimeric antigen receptor (CAR)-containing cells is provided, the method including: obtaining platelets from blood; obtaining mitlets from the platelets; co-incubating the mitlets with CAR-containing cells, wherein uptake of the mitlets by the CAR-containing cells enhances metabolic activity of the CAR-containing cells; and preparing the enhanced CAR-containing cells for patient treatment of an indication. In some embodiments, the indication includes a tumor. In some embodiments, the tumor includes a solid tumor. In some embodiments, the tumor includes a non-solid tumor.

In some embodiments, the blood is from the patient. In some embodiments, the blood is from donors. In some embodiments, the donors include family members of the patient.

In some embodiments, the obtaining of the platelets includes: adding an anticoagulant and a buffer to the blood to form a mix; separating the mix into supernatant and platelet rich plasma (PRP); and collecting the PRP. In some embodiments, the anticoagulant includes anticoagulant citrate dextrose (ACD). In some embodiments, the techniques described herein relate to a method or claim 9, wherein the buffer includes Tyrode's buffer. In some embodiments, Tyrode's buffer has a pH in a range from 6.0 to 7.0.

In some embodiments, the obtaining of the mitlets includes: stimulating the collected PRP, thereby expelling extracellular vesicles from the platelets in the PRP; and collecting the extracellular vesicles, wherein the collected extracellular vesicles include mitochondria, wherein the mitlets include the collected extracellular vesicles. In some embodiments, the stimulating of the PRP includes exposing the PRP to immune complexes in the presence of Ca2+. In some embodiments, the immune complexes include heat-aggregated IgG. In some embodiments, a concentration of heat-aggregated IgG is from 0.1 mg/mL to 2.5 mg/mL.

In some embodiments, the method further includes matching mtDNA haplotypes of the obtained mitlets and mtDNA haplotypes of the patient.

In some embodiments, the obtaining of the mitlets step further includes expanding the mitlets in a bioreactor. In some embodiments, the method further includes coating the mitlets following the expanding step. In some embodiments, the co-incubating step occurs during manufacturing of the CAR-containing cells.

In some embodiments, the preparing step includes: adding a cryopreservative to the mitlets and the enhanced CAR-containing cells; and freezing the mitlets and the enhanced CAR-containing cells. In some embodiments, the cryopreservative is selected from the group consisting of a saccharide, an oligosaccharide, and a polysaccharide. In some embodiments, the cryopreservative includes DMSO. In some embodiments, the cryopreservative includes trehalose. In some embodiments, the cryopreservative includes phosphate buffered saline.

In some embodiments, the method further includes administering a therapeutically effective amount of the mitlets to the patient following the preparing step. In some embodiments, the administering step further includes administering a therapeutically effective amount of the enhanced CAR-containing cells to the patient. In some embodiments, the administering of the CAR-containing cells precedes the administering of the mitlets. In some embodiments, the administering of the CAR-containing cells and the administering of the mitlets to the patient occur concurrently. In some embodiments, the administering step further includes administering a follow-up amount of the mitlets after at least two days following the initial administering step. In some embodiments, the administering step is repeated after every two days.

In yet another embodiment, a method of treating an indication is provided, the method including: administering therapeutically effective amounts of mitlets and CAR-containing cells to the patient to treat the indication, wherein the mitlets are obtained from a source. In some embodiments, the indication includes a tumor. In some embodiments, the tumor includes a solid tumor. In some embodiments, the tumor includes a non-solid tumor. In some embodiments, the source is placental tissue. In some embodiments, the source is bone marrow. In some embodiments, the source is adipose tissue. In some embodiments, the source is platelets obtained from blood. In some embodiments, the mitlets were expanded in a bioreactor.

In some embodiments, the mitlets includes a coating. In some embodiments, the coating includes an asialoglycoprotein (AsG). In some embodiments, the AsG includes asialoorosomucoid (AsOR). In some embodiments, the coating further includes poly-L-lysine linked to the AsOR. In some embodiments, the coating is complexed with a conjugate. In some embodiments, the conjugate includes AsOR. In some embodiments, the conjugate further includes listeriolysin O (LLO).

In some embodiments, the administering of the mitlets occurs prior to the administering of the CAR-containing cells. In some embodiments, during the administering step, the mitlets are co-administered with the CAR-containing cells. In some embodiments, the method further includes, at least two days after the administering step, administering a follow-up dose of mitlets to the patient. In some embodiments, the method further includes, at least every two days after the administering step, administering repeat doses of mitlets to the patient.

In some embodiments, the CAR-containing cells include CAR-T cells. In some embodiments, the CAR-containing cells include CAR-NK cells.

In yet another embodiment, a formulation for treatment of an indication is provided, the formulation including: mitlets including mitochondria; and cells expressing a CAR, wherein the mitlets are from a source. In some embodiments, the CAR-expressing cells are T cells. In some embodiments, the CAR-expressing cells are NK cells. In some embodiments, the indication includes a tumor. In some embodiments, the tumor includes a solid tumor. In some embodiments, the tumor includes a non-solid tumor. In some embodiments, the source includes placental tissue. In some embodiments, the source includes bone marrow. In some embodiments, the source includes adipose tissue. In some embodiments, the source includes platelets. In some embodiments, the platelets are obtained from blood. In some embodiments, the mitlets are expanded in a bioreactor.

In some embodiments, the techniques described herein relate to a formulation, further including a cryopreservative. In some embodiments, the cryopreservative is from the group consisting of a saccharide, an oligosaccharide, and a polysaccharide. In some embodiments, the cryopreservative includes DMSO. In some embodiments, the mitlets and the cells expressing the CAR are held into separate containers. In some embodiments, the containers include cryopreservation bags. In some embodiments, the containers are made from fluorinated ethylene propylene (FEP). In some embodiments, the containers are made from ethylene vinyl acetate (EVA). In some embodiments, the contains are made from polyolefin.

The methods and formulations disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. One or more of the disclosed steps may be repeated any number of times. After considering this discussion, and particularly after reading the section entitled “Detailed Description,” one will understand how the features of the methods disclosed herein provide desirable attributes over other known devices and methods.

It is to be understood that any features of the method disclosed herein may be combined together in any desirable manner. Moreover, it is to be understood that any combination of features of this method may be used together, and/or may be combined with any of the examples disclosed herein. Still further, it is to be understood that any feature or combination of features of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows a graphical representation of a schema related to CAR immunotherapy from collection of immune cells from a patient to infusion of engineered autologous immune cells to the patient.

FIGS. 2A-2B show a flowchart showing a method according to some of the embodiments.

FIG. 3 shows a flowchart of collecting and preparing mitlets for transplantation according to some embodiments of the invention.

FIGS. 4A and 4B depicts mitlets according to some embodiments of the invention, wherein FIG. 4A shows mitlet expulsion from platelets and FIG. 4B shows mitlet uptake in different cells.

FIG. 5 shows a method of growing multiple types of mitochondria and the coating thereof for transport and transfusion according to some embodiments of the invention.

FIG. 6 shows a graphical representation of combination therapy including administration of mitlets at various stages of immunotherapy involving CAR cells according to some embodiments of the invention.

FIG. 7 is a dot plot that represents mitlet populations where the mitlets, labeled with DsRed, are represented as approximately 40% of the total CD41+ cells.

FIGS. 8A and 8B are confocal fluorescent images that depict mitlet uptake by immunological cells according to some embodiments of the invention, wherein FIG. 8A shows mitlet uptake after 60 minutes, and FIG. 8B shows mitlet uptake after 24 hours.

FIG. 9 is a confocal fluorescent image that depicts mitlet uptake by bone marrow tissue in as little as 15 minutes according to some embodiments of the invention.

FIGS. 10A and 10B are graphs showing cellular uptake of mitlets as determined by fluorescent-based cellular tracking assays according to some embodiments of the invention, wherein FIG. 10A shows cellular uptake of mitlets in neutrophils and FIG. 10B shows cellular uptake of mitlets in leukocytes.

FIG. 11 is a confocal fluorescent image that depicts mitlet uptake by T cells. The blue-labeled elements in the image represent the nucleus of the T cell. The green-labeled elements in the image represent the cellular membrane of the cells. And the red-labeled elements represent the mitlets.

FIGS. 12A and 12B show the effects of mitlets, according to some embodiments of the invention, on cytokine storm induced in a murine model via H1N1 infection, wherein FIG. 12A shows the measured concentration of IL-6 plasma levels pre- and post-infection with H1N1 among different treatment groups, and FIG. 12B shows the post-infection survival rate among different treatment groups.

FIG. 13 shows the survival rate of different treatment groups of mice that underwent induced sepsis according to some embodiments of the invention.

FIGS. 14A-14D show survival rates of different treatment groups where mitlets isolated from young mice or isolated liver mitochondria from young mice were administered to aged recipient mice with induced sepsis infections. FIGS. 14A and 14B show results from experiments with mitlet administration and FIG. 14C shows combined results of FIGS. 14A and 14B. FIG. 14D show results from administration of isolated liver mitochondria.

FIGS. 15A-15D show results indicating significantly reduced cytokine IL-6 and bacterial levels after mitlet administration. FIG. 15A shows bacterial levels following mitlet administration in a first experiment. FIG. 15B shows bacterial levels following mitlet administration in a second experiment. FIG. 15C shows bacterial levels from a combination of FIGS. 15A and 15B. FIG. 15D shows plasma IL-6 levels from the first experiment.

FIG. 16 shows flow cytometry cell-sorting results of isolating neutrophils according to some embodiments of the invention. Following the cell-sorting, 66.4% of the sorted cells were CD11b⁺ and Ly6G⁺.

FIG. 17 shows flow cytometry cell-sorting results of isolating monocytes according to some embodiments of the invention. Following the cell-sorting, 84.5% of the sorted cells were CD11b⁺ and Ly6C⁺.

FIG. 18 shows flow cytometry cell-sorting of isolating T cells according to some embodiments of the invention. Following the cell-sorting, 80% of the sorted cells were CD3⁺.

FIG. 19 shows flow cytometry cell-sorting of the CD3⁺ cells according to some embodiments of the invention. Following the cell-sorting, 64% of the sorted cells were CD4⁺ and 28% of the sorted cells were CD8⁺.

FIG. 20 shows flow cytometry cell-sorting of isolating NK cells according to some embodiments of the invention. Following the cell-sorting, 74.2% of the sorted cells were CD49⁺ and CD3⁻.

FIGS. 21A and 21B shows the bioenergetic profiling of immune cells according to some embodiments of the invention following co-incubation of mitlets after 6 hours as measured by the oxygen consumption rate (OCR), wherein FIG. 21A shows the OCR of bone marrow neutrophils and FIG. 21B shows the OCR of T cells.

FIGS. 22A and 22B shows some embodiments of the bioenergetic profiling as measured by the OCR of bone marrow neutrophils and T cells following vehicle or mitlet co-incubation, wherein FIG. 22A shows the basal OCR and FIG. 22B shows the maximal OCR.

FIG. 23 shows the bioenergetic profiling of resting and activated T cells according to some embodiments of the invention.

FIGS. 24A and 24B show the bioenergetic profile of T cells as measured by OCR according to some embodiments of the invention. FIG. 24A shows resting T cells in the presence of various concentrations of mitlets (number of mitlets per cell) after 24 hours. FIG. 24B shows activated T cells in the presence of various concentrations of mitlets (number of mitlets per cell) and mitos.

FIGS. 25A and 25B shows the bioenergetic profiling of T cells resting and activated T cells following absorption of mitlets as measured by OCR according to some embodiments of the invention. FIG. 25A shows the basal OCR and FIG. 25B shows the maximal OXR.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

Sepsis, covid, cancer, and many other diseases tend to be more severe for the elderly because the immune system weakens with age. A new method of treatment has been developed to reverse immune weakness by making older immune systems at least temporarily young again. Injection of recently-discovered immune components called mitlets into patients for treatment of various diseases or conditions. Mitlets can be viewed as a type of biological “battery pack” that platelets, T cells, NK cells, neutrophils, and other immune components exchange to conserve energy. “Young” mitlets are able to be grown in bioreactors specialized for their growth and expansion and then injected into the body, where these immune cells readily uptake them and use the mitochondria inside the mitlets. In early tests, mammalian models of diseases that receive these injections gained immune strength, reduced cytokine storms, and dramatically improved survival against bacterial and viral infections. The enhanced energetics provided by the mitlets persist at least several weeks. ISET (Immune System Energetic Transplantation) therapy is believed to be compatible with other immunotherapies such as CAR-T, monoclonal antibodies, and checkpoint inhibitors.

ISET therapy replaces the “battery packs” of the immune system, so it works faster and fights back more effectively against infection—in effect, making an old immune system temporarily young again. In some embodiments, ISET therapy includes growing large numbers of special extracellular vesicles called mitlets in external bioreactors, and then transfusing them into the patient's bloodstream. In some embodiments, ISET therapy includes collection large numbers of mitlets from platelets from blood donors. The mitlets are almost instantly absorbed by the other immune components floating in the blood, such as neutrophils, T cells, NK cells, and platelets, which enhances or improves the metabolic capacities of these respective components, which allows them to improve their respective cellular capabilities.

ISET not only provides the immune system with supplemental mitochondria but can provide the elderly younger mitochondria as well. In elderly people, mitochondria tend to decline in metabolic capacity, thereby reducing their ability to fully meet the cell's metabolic requirements. ISET therapy can grow younger, more powerful mitochondria included in mitlets (sourcing them from seed materials from young donors). These younger mitochondria, when transplanted into elderly immune system, gets absorbed by the various immunological components in the elderly immune system, thereby “combining” with the existing mitochondria to enhance the metabolic capacity of the cell, which makes the immune system more powerful (i.e., “younger”).

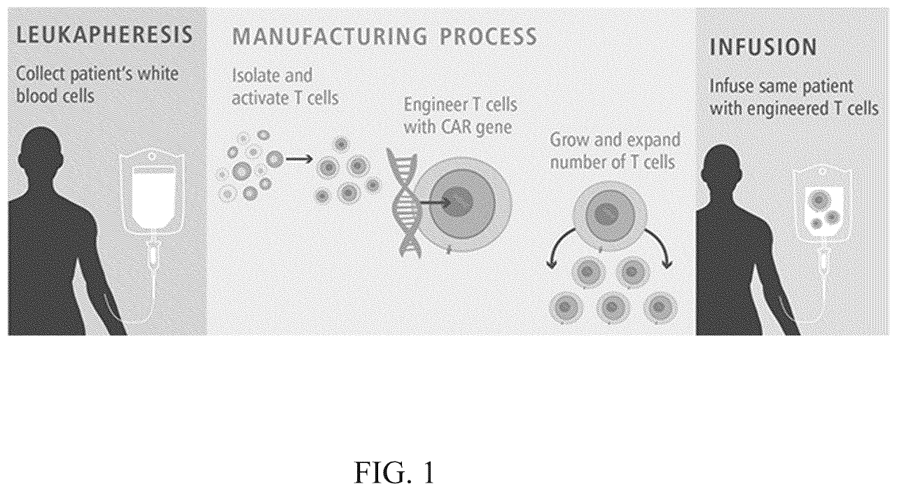

As shown in FIG. 1, the process of CAR-T cell therapy begins with collecting peripheral blood from a patient and performing leukapheresis to isolate and extract peripheral mononuclear blood cells (“PBMC”) including white blood cells. More recently, umbilical cord blood (a product from childbirth) may serve as another source of immunological cells. After leukapheresis is performed, T cells are isolated from the collected immunological cells (e.g., white blood cells) and then activated ex vivo by a variety of different techniques that include, but are not limited to, cell-based activation, bead-based activation (e.g., antibody-coated magnetic beads, antibody-coated nanobeads, and the like), soluble and dissociable T-cell stimulation reagents (e.g., Expamer), and anti-CD3 antibodies. Wang X and Rivière I, “Clinical manufacturing of CAR T cells: foundation of a promising therapy” Mol Ther Oncolytics, 2016 Jun. 15; 3:16015. As shown in FIG. 1, the immunological cells are genetically modified to express the CAR after delivery by viral or non-viral delivery systems. Some examples of viral delivery systems include γ-retroviral vectors and lentiviral vectors, with the latter being more commonly used due to their safer integration site profile. Some examples of non-viral delivery systems include transposon/transposase-based systems and mRNA electroporation.

Following CAR expression in immunological cells, the CAR-expressing immunological cells are grown and expanded. For these CAR-expressing cells, there are several platforms that can be used for expansion. One non-limiting example includes expansion of CAR-containing cells using bioreactors. Another non-limiting example is the Miltenyl CliniMACS Prodigy® system, which includes a cell washer, a magnetic cell separation system, and a cell cultivation device. CAR-T cells and CAR-NK cells isolated from immunological cells have been expanded using the CliniMACS Prodigy® system. Another non-limiting example for expansion of CAR-containing cells includes recursive artificial antigen-presenting cells (AAPC) stimulation, wherein expansion of CAR-containing cells generated by the transposon/transposase-based system undergo recursive stimulation with γ-irradiated AAPCs in the presence of stimulatory molecules such as cytokines (e.g., IL-2 and IL-21) in one non-limiting example. In some examples, engineered cell lines such as K652 express a variety of stimulatory molecules such as CD40, CD40L, CD70, CD80, CD83, CD86, CD137L, ICOSL, GITRL, and CD134L. Wang X and Rivière I, Mol Ther Oncolytics, 2016 Jun. 15; 3:16015. Following expansion, CAR-containing cells are formulated using various washes and formulations. Further, the formulated CAR-containing cells may be preserved for transport via freezing (e.g., cryopreservation). An example of a component used in a cryopreservation medium includes dimethyl sulfoxide (DMSO).

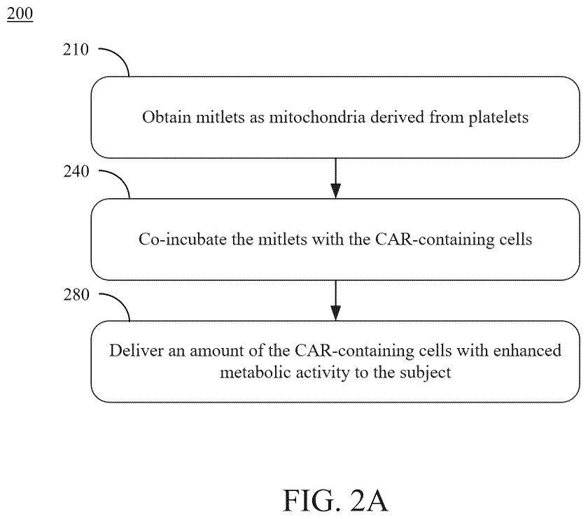

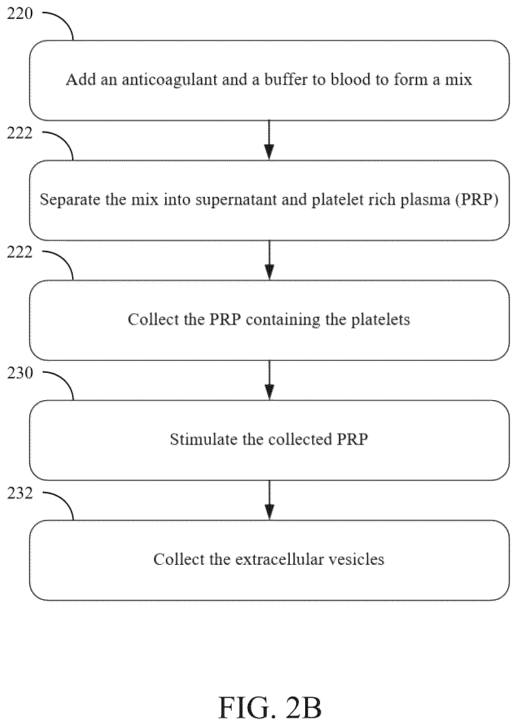

To reduce some of the limitations associated with CAR-based therapies, a method of enhancing activity of CAR-containing cells is provided as shown in FIG. 2A, the method comprising: obtaining mitlets as mitochondria derived from platelets in step 210; co-incubating the mitlets with CAR-containing cells in step 240, wherein uptake of the mitlets by the CAR-containing cells enhances metabolic activity of the CAR-containing cells; and delivering an amount of the CAR-containing cells with enhanced metabolic activity to the subject for treatment of an indication in step 280. In some embodiments, the indication comprises a tumor. In some embodiments, the tumor comprises a solid tumor. In some embodiments, the tumor comprises a non-solid tumor.

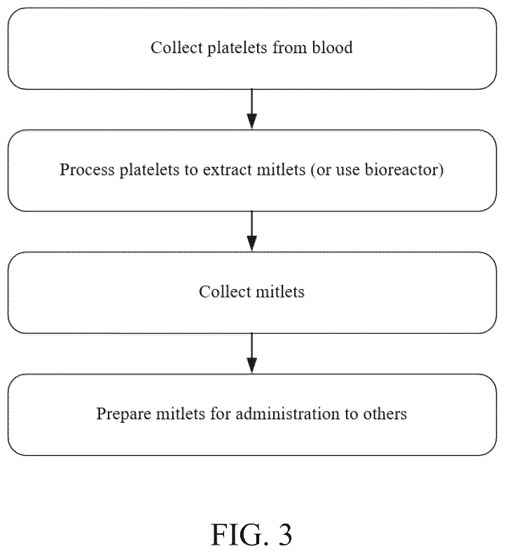

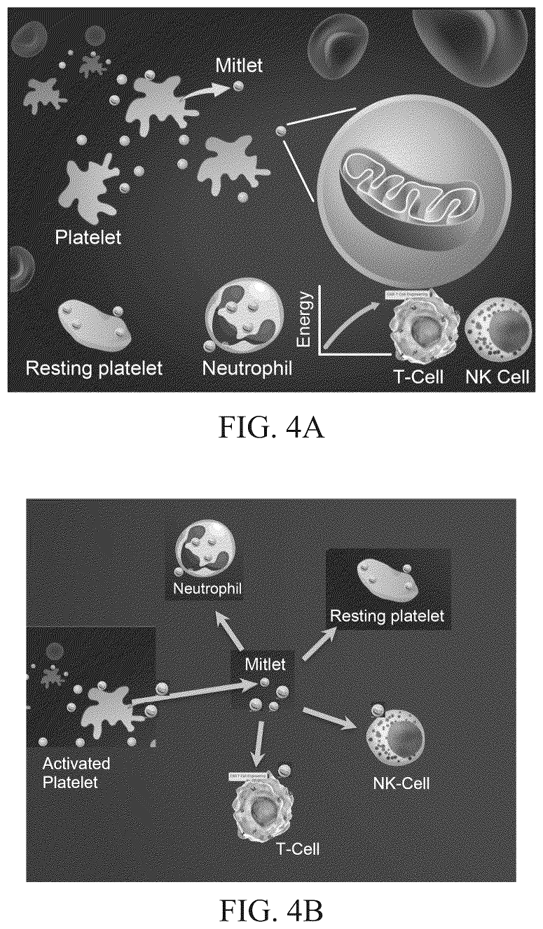

As shown in FIG. 2B, in some embodiments, the step of obtaining the platelets comprises: adding an anticoagulant and a buffer to the blood to form a mix in step 220; separating the mix into supernatant and platelet rich plasma (PRP) in step 222; and collecting the PRP in step 224. In some embodiments, the step of obtaining the mitlets comprises stimulating the collected PRP in step 230, thereby expelling extracellular vesicles from the platelets in the PRP; and collecting the extracellular vesicles in step 232, wherein the collected extracellular vesicles comprise mitochondria, and wherein the mitlets comprise the collected extracellular vesicles. As shown in FIG. 3, in some embodiments, platelets are obtained from the blood of donors or the patient. In some embodiments, the donors comprise family members of the patient. In some embodiments, the blood is from mice. Mouse blood is used to test the feasibility of the method of extracting the mitlets. In some embodiments, the blood is from human donors.

In some embodiments, adding the anticoagulant and the buffer to the blood prevents the blood from becoming thick and solid. In some embodiments, the anticoagulant is ACD (20%). In some embodiments, the buffer is 40% Tyrode's buffer pH 6.5. After adding the anticoagulant and the buffer to blood, the mixture is then separated into PRP. In some embodiments, the separating is by centrifuging. Plasma is the liquid portion of whole blood. It is composed largely of water and proteins, and it provides a medium for red blood cells, white blood cells and platelets to circulate through the body. Platelets are blood cells that cause blood clots and other necessary growth healing functions. After the centrifuging of the mixture, blood cells formed a pellet that accumulates at the bottom of a tube. The pellet is referred to as PRP, which contains concentrated platelets.

In some embodiments, buffers are then added to the collected PRP to resuspend the platelets. The platelets are then activated or stimulated. In some embodiments, to activate platelets, any number of substances can be used including carbon radioisotopes, prostaglandins, serotonin, adenosine triphosphate, collagen, 1-lactate dehydrogenase, thrombin, magnesium, adenosine, calcium, heat-aggregated antibodies, or any combination thereof. In some embodiments, platelets are activated by freeze-thaw cycles. As used herein, the term “freeze-thaw cycle” refers to freezing of the mitochondria of some the embodiments to a temperature below 0° C., maintaining the mitochondria in a temperature below 0° C. for a defined period of time, and thawing the mitochondria to room temperature or body temperature or any temperature above 0° C. The term “room temperature”, as used herein, refers to a temperature of between 18° C. and 25° C. The term “body temperature”, as used herein, refers to a temperature of between 35.5° C. and 37.5° C., preferably 37° C.

Finding a source of mitochondria to transplant is a challenge. Just like any donated organ, mitochondria from young healthy donors are in short supply. Some diseases or injuries might be cured by autologous mitochondria removed from a leg muscle in one's own body for example—however, for many other diseases, the “patients” have poor quality mitochondria due to age or mutation to mitochondrial DNA (mtDNA). For these patients, donated mitochondria are a preferred solution. In addition, freshly fully isolated mitochondria die quickly within minutes of isolation and may also provoke immune reactivity when put naked into the bloodstream, thus reducing their effectiveness as a therapy. To overcome these obstacles, it would be convenient to find a readily accessible source of donation-ready mitochondria, which at the same time, that are coated or encased in vesicle that reduce detection or reactivity by the immune system.

A platelet from human blood contains 4-5 mitochondria on average that are expelled in extracellular vesicles when platelets are activated. In some embodiments, the platelet-derived mitochondria-containing extracellular vesicles are referred to mitlets herein. These mitlets are usually larger (>400 nM), and less well-known than other platelet extracts or lysates (30-100 nM), however other sizes may also apply.

Mitlets have been shown to donate mitochondria to cells nearby, which can increase the respiratory activity of the cells that absorb them, thus improving the cellular respiration (and metabolic capabilities) of the cells that uptake the mitlets. Mitlets obtained from platelets include several desirous properties, any one of the following or a combination thereof, make mitlets suitable for fast commercialization: Mitlets can be extracted from donated platelets that have “expired” and must be thrown away; mitlets represent another good medically-valid use for platelets which otherwise might go to waste; mitlets can be collected at most blood banks, who already have all the needed skilled personnel, clean handling practices, and equipment needed, and are already in close proximity to hospitals, thus making mitlet products potentially available to world-wide use extremely soon. In some embodiments, millets may be included in a variety of platelet transfusions and therefore are more likely to be embraced and tested by medical professionals who are already familiar with blood transfusion therapies.

In some embodiments, the blood is derived from a mammalian subject. In some embodiments, the mammalian subject is a human subject. In some embodiments, the mammalian subject is selected from a group consisting of: a human, a horse, a dog, a cat, a mouse, a rat, a cow and a sheep. Each possibility represents a separate embodiment of the present invention.

In some embodiments, mitlets include mitochondria and a coating on the mitochondria. In some embodiments, the mitlets are derived from a mammalian cell. In some embodiments, the mammalian cell is a human cell. In some embodiments, the mitlets are derived from cells in culture. In some embodiments, the mitlets are derived from a tissue. In some embodiments, the mitlets are derived from a cell or a tissue selected from the group consisting of: human placenta, human placental cells grown in culture and human blood cells. In some embodiments, the mitlets are derived from a cell or a tissue selected from the group consisting of: placenta, placental cells grown in culture and blood cells. In some embodiments, the mitlets are derived from adipose tissue.

In some embodiments, the step of obtaining the mitlets comprises selecting a source comprising mitochondria and stem cells. In some embodiments, the source comprises placenta tissue comprising the mitochondria and the stem cells. In some embodiments, the source comprises cells of the bone marrow comprising the mitochondria and the stem cells. In some embodiments, the source comprises adipose tissue comprising the mitochondria and the stem cells.

In some embodiments, the step of obtaining the mitlets further comprises extracting the stem cells and the mitochondria from the source and segregating the stem cells and the mitochondria into first and second pools respectively. In some embodiments, the extraction step comprises segregating stem cells and miscellaneous mitochondria and isolating these cells into two different pools of material. In some embodiments, the first pool of material includes the stem cells. In some embodiments, the stem cells are isolated prior to the miscellaneous mitochondria. In some embodiments, the second pool includes miscellaneous mitochondria that is extracted from the remainder of the tissue from which stem cells were previously extracted. In some embodiments, the remainder of the tissue is grounded up prior to extracting the miscellaneous mitochondria. In some embodiments, the tissue includes umbilical cord blood. In some embodiments, the tissue includes an umbilical cord. In some embodiments, the tissue includes bone marrow. In some embodiments, the tissue includes adipose tissue. In some embodiments, the tissue includes any tissue that is associated with stem cells. In some embodiments, the tissue includes any stem-cell including tissue.

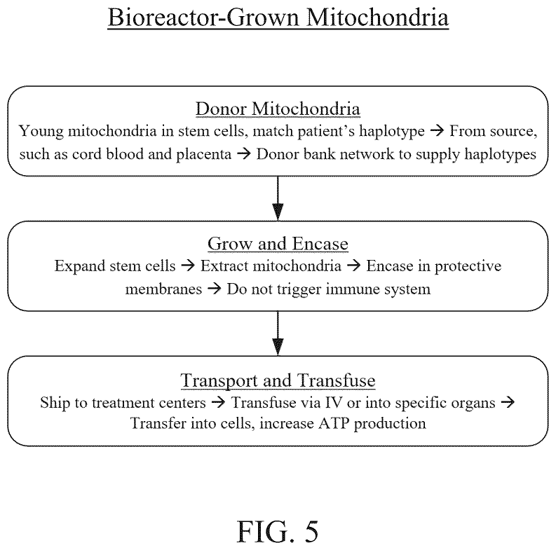

In some embodiments, the step of obtaining mitlets further comprises a step of growing the mitochondria in the bioreactor, the growing of the mitochondria including incubating (i.e., packing) the miscellaneous mitochondria into the stem cells and expanding the packed stem cells in the bioreactor as shown in FIG. 5. In some embodiments, the miscellaneous mitochondria not used in the stem cells can be used for therapeutics. In some embodiments, the step of obtaining the mitlets further includes expanding the stem cells packed with the miscellaneous mitochondria at the highest rate allowed by the bioreactor. In some embodiments, the step of obtaining the mitlets further includes adjusting selection conditions to favor high quality mitochondria. In some embodiments, the selection conditions include hypoxia, glucose starvation, use of a Treefrog process, or any combination thereof. In some embodiments, the step of growing the mitochondria in the bioreactor further includes differentiating the remaining stem cells into megakaryocytes, thereby increasing the numbers of mitochondria being produced a hundredfold, even a thousandfold, over traditional cell culture techniques. Megakaryocytes are polyploid cells derived from hematopoietic stem cells that are found in the bone marrow. The role of megakaryocytes includes generation of blood platelets that participate in localized clot formation to block hemorrhages. Megakaryocytes that are grown in the bioreactor can come from not only hematopoietic stem cells found in the bone marrow but from other sources.

In some embodiments, megakaryocytes are generated/induced from stem cells that are sourced from adipose tissue. In some embodiments, the adipose tissue comprises subcutaneous adipose tissue. In some embodiments, the method further includes obtaining the source. In some embodiments, the source comprises adipose tissue, wherein the adipose tissue is obtained by any known technique in the art. In some embodiments, after obtaining the adipose tissue, the method further includes digesting the adipose tissue with a digestion agent. In some embodiments, the digestion agent is collagenase type II. In some embodiments, the method further includes centrifuging the digested adipose tissue to generate an adipose-derived mesenchymal stromal stem cell line (ASCL). In some embodiments, the method further includes treating the ASCL with megakaryocyte lineage induction media to generate the megakaryocytes. In some embodiments, the megakaryocyte lineage induction media includes: 2 mM L-glutamine; 100 U/mL penicillin-streptomycin solution; 0.5% bovine serum albumin (BSA); 4 μg/mL LDL cholesterol; 200 μg/mL iron-saturated transferrin; 10 μg/ml insulin; 50 μM 2-β-mercaptoethanol, nucleotides (about 20 μM for each of ATP, UTP, GTP, and GTP), and 50 ng/ml thrombopoietin (TPO) in Iscove's Modified Dulbecco's Medium (IMDM). See also U.S. Pat. No. 10,113,147.

In some embodiments, the megakaryocytes are generated/induced from stem cells that are sourced from pluripotent stem cells (PSCs). In some embodiments, the step of obtaining the mitlets further includes obtaining PSCs. In some embodiments, the method further includes transducing expression of transcription factors in the PSCs via a vector. In some embodiments, the transcription factors were cloned into a vector backbone. In some embodiments, the vector comprises a lentiviral vector. In some embodiments, the transcription factors include GATA binding protein 1 (GATA1); friend leukemia integration 1 (FLI1); and T-cell acute lymphocytic leukemia protein 1 (TAL1). See also Moreau T, et al. “Large-scale production of megakaryocytes from human pluripotent stem cells by chemically defined forward programming.” Nat Commun. 2016; 7:11208. In some embodiments, the method further includes maintaining the transduced PSCs in a PSC medium for about 2 days. In some embodiments, the PSC medium includes fibroblast growth factor (FGF2) and Activin-A. In some embodiments, the method further includes maintaining the transduced PSCs in megakaryocyte medium. In some embodiments, the megakaryocyte medium includes TPO and SCF for at least 5 days after the maintenance in the PSC medium step.

In some embodiments, the method further includes the step of incubating/transferring the extracted mitochondria into the extracted stem cells to produce packed stem cells. In some embodiments, the method further includes expanding the packed stem cells in a bioreactor. In some embodiments, the method further includes adjusting conditions of an environment of the bioreactor to favor growth of the mitochondria of the packed stem cells. In some embodiments, the method further includes converting the packed stem cells into megakaryocytes. In some embodiments, the method further includes isolating the mitochondria from the megakaryocytes. In some embodiments, the method further includes applying a coating to the mitochondria following isolation thereof from the megakaryocytes.

In some embodiments, the method further includes matching mtDNA haplotypes of the obtained mitlets and mtDNA haplotypes of the patient at least to a degree in which the patient does not develop an acute immune response against the mitlets.

In some embodiments, the step of obtaining the mitlets step further comprises expanding the mitlets in a bioreactor. In some embodiments, the method further includes coating the mitlets. In some embodiments, a coating step follows the expanding step. In some embodiments, the coating comprises asialoorosomucoid (AsOR). In some embodiments, the coating further comprises poly-L-lysine (PL). In some embodiments, the method further includes complexing the coated mitlets with a conjugate. In some embodiments, the conjugate includes the AsOR. In some embodiments, the conjugate further includes listeriolysin O (LLO).

In some embodiments, the administering of the mitlets occurs prior to the administering of the CAR-containing cells. In some embodiments, during the administering step, the mitlets are co-administered with the CAR-containing cells. In some embodiments, the method further includes administering a follow-up dose of mitlets to the patient at least two days after the administering of the CAR-containing cells. In some embodiments, the method further includes, at least every two days after the administering step, administering repeat doses of mitlets to the patient.

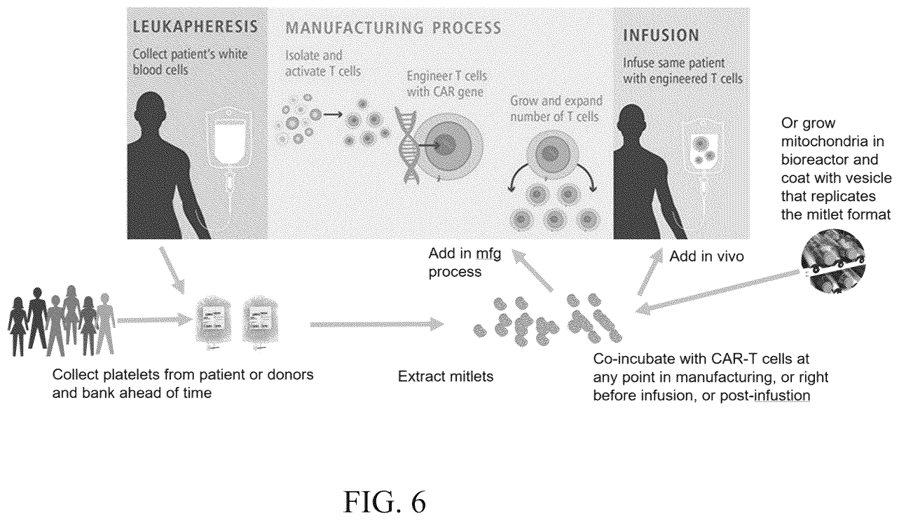

In some embodiments, mitlets are added during the manufacturing process of the CAR-containing cells as shown in FIG. 6 as a co-incubating step. In some embodiments of the method, the co-incubating step is performed during manufacturing of the CAR-containing cells. In some embodiments of the method, the co-incubating step is performed during the growth and expansion of cells expressing the CAR gene as shown in FIG. 6. In some embodiments of the method, the co-incubating step is performed during activation of the T cell. In some embodiments of the method, the co-incubating step is performed during engineering the T cells with the CAR gene.

In some embodiments, the preparing step includes adding cryopreservative to the mitlets and the enhanced CAR-containing cells. In some embodiments, the cryopreservative is selected from the group consisting of a saccharide, an oligosaccharide, and a polysaccharide. In some embodiments, the cryopreservative comprises DMSO. In some embodiments, the cryopreservative comprises trehalose. In some embodiments, the preparing step further includes freezing the mitlets and the enhanced CAR-containing cells.

In some embodiments, the method further includes administering a therapeutically effective amount of the mitlets to the patient following the preparing step. In some embodiments, the administering step further includes administering a therapeutically effective amount of the enhanced CAR-containing cells to the patient. In some embodiments, the administering of the CAR-containing cells precedes the administering of the mitlets. In some embodiments, the administering of the CAR-containing cells and the administering of the mitlets to the patient are performed concurrently. In some embodiments, the administering step further comprises administering a follow-up dose of the mitlets after at least two days following the initial administering step. In some embodiments, the administering step is repeated after every two days following the initial administrating step.

In another embodiment, a method of treating an indication is provided, the method including the steps of administering a therapeutically effective amount of mitlets and a therapeutically effective amount of CAR-containing cells to the patient to treat the indication. In some embodiments, the indication includes a tumor. In some embodiments, the tumor includes a solid tumor. In some embodiments, the tumor includes a non-solid tumor.

In some embodiments, the mitlets are obtained from a source. In some embodiments, the source is placental tissue. In some embodiments, the source is bone marrow. In some embodiments, the source is adipose tissue. In some embodiments, the source is platelets obtained from blood.

In some embodiments, the mitlets are collected from expanded cells or cellular fragments in a bioreactor. Platelets are produced by megakaryocytes in the bone marrow. Because mitlets may be collected from expelled vesicles from activated platelets, growth and expansion of platelets in a bioreactor provides the ability to grow and collect mitlets at scale. An example of using protoplatelets for platelet production in bioreactors has been described. Thon et al. “Platelet bioreactor: accelerated evolution of design and manufacture,” Platelets 2017 July; 28(5):472-477.

In some embodiments, the CAR-containing cells comprise CAR-T cells. In some embodiments, the CAR-containing cells comprise CAR-NK cells.

In some embodiments, CAR-containing cells and mitlets are stored in separate containers. In some embodiments, the CAR-containing and mitlets are combined into a same container prior to administration into a patient. In some embodiments, the mitlets and CAR-containing cells are co-administered at the same administration site of a patient. In some embodiments, the administration of the CAR-containing cells and the mitlets are staggered at the same administration site, wherein the CAR-containing cells are administered prior to the mitlets (or vice versa). In some embodiments, the administration site of the CAR-containing cells and the mitlets differ with respect to each other. In some embodiments, wherein the containers comprise bags. In some embodiments, the bags are cryopreservation bags. In some embodiments, the bags comprise fluorinated ethylene propylene (FEP). In some embodiments, the bags comprise ethylene vinyl acetate (EVA). In some embodiments, the bags comprise polyolefin.

The following examples are presented to provide a more complete understanding of the invention. The specific techniques, conditions, materials, proportions and reported data set forth to illustrate the principles of the invention are exemplary and should not be construed as limiting the scope of the invention.

### Example 1

This example illustrates how mitlets that include vesicles and mitochondria encapsulated in the vesicles are collected from mammalian blood according to some embodiments.

The following steps were performed:

1. Blood was collected by from mouse donors. In this specific protocol, the donors were male DsRed mice, which are transgenic mice that express the red fluorescent protein variant DsRed. MST under the control of the chicken beta actin promoter coupled with the cytomegalovirus (CMV) immediate early enhancer. Here, 3×1 mL (1 mL/mouse) of blood was used.

2. ACD (20%) was added as an anticoagulant and 40% Tyrode's buffer pH 6.5 was also added to the blood. The blood mixture (20% ACD+40% Tyrode's buffer (TB) pH 6.5) was then centrifuged for 3 min at 500 g. PRP and buffy coat then collected and centrifuged for 2 min at 300 g.

3. PRP was collected and 20% ACD+10 mM EDTA added before a centrifugation step of 5 min at 1 300 g.

4. Each pellet was suspended in 0.1 mL TB pH 6.5 and 0.9 mL of TB pH 7.4 was added.

5. Platelets were pooled and counted using a cellometer and diluted at 10e8/mL in TB 7.4.

6. 900 millions of platelets were obtained in total and 5 mM CaCl₂) was added prior stimulation.

7. Platelets were stimulated overnight (16 h) at room temperature with heat aggregated-IgG at 0.5 mg/mL. Heat aggregated-IgG was prepared by aggregating human IgG (25 mg/mL, MPBIO) at 62° C. for 1 hour.

8. 10 mM EDTA was added to stop the stimulation.

9. Stimulated-platelets were centrifuged at 300 g for 5 min to remove remnant platelets or cells.

10. Supernatant was collected and mitlets were analyzed using a flow cytometer.

11. Remaining platelets were evaluated and represented less than 1% contamination.

12. Obtained mitlets were diluted 3 times with PBS and centrifuged at 18 000 g for 90 minutes at 18° C.

13. Pellet was resuspended in 0.3 mL PBS and mitlets were counted by flow cytometry. Concentration was estimated at 1.5×10⁹ mitlets/ml.

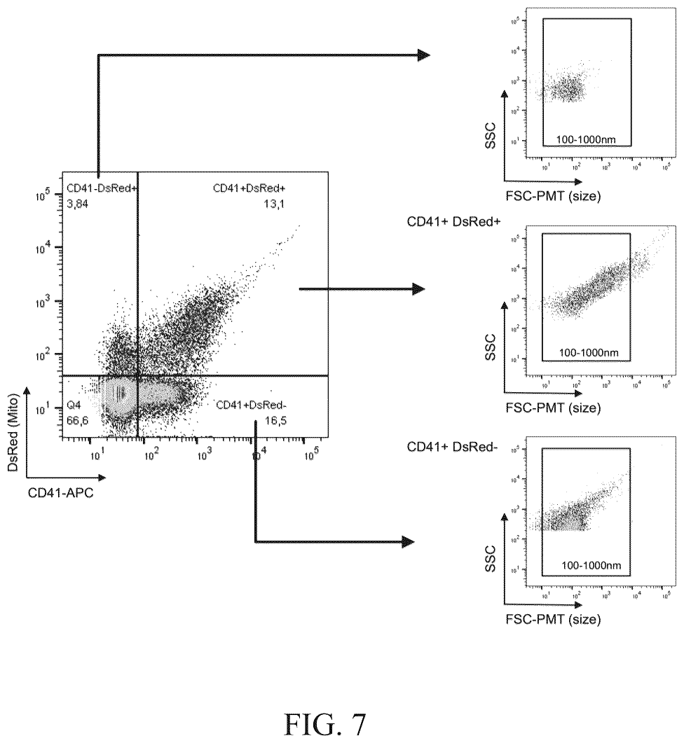

14. Mitlets may be tagged with CD41 tags to enable them to be counted in a flow cytometer. If so tagged, mitlets represented approximately 40% of the total CD41+PEVs. Dotplot representing mitlet populations are illustrated in FIG. 7. (DsRed=mitlets).

### Example 2

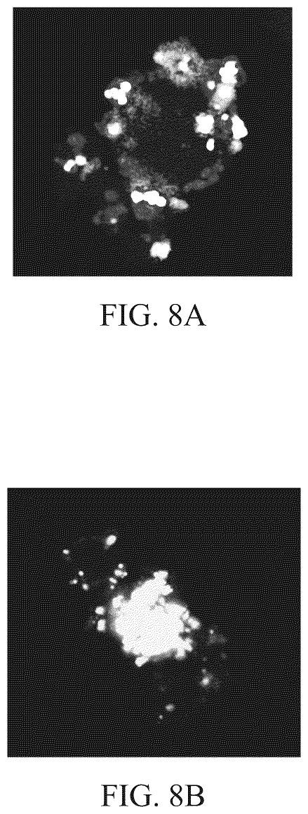

Mouse monocytes were isolated from bone marrow and incubated with mouse mitlets over at least 24 hours. The mouse mitlets were generated from platelet activation in transgenic mice expressing fluorescent protein (e.g., DsRed) in mitochondria. Results shown in FIG. 8A demonstrate isolated monocytes absorbing the mitlets in as little as 60 minutes. Over 24 hours, the isolated monocytes absorb a significant amount of mitochondria from the mitlets as shown in FIG. 8B.

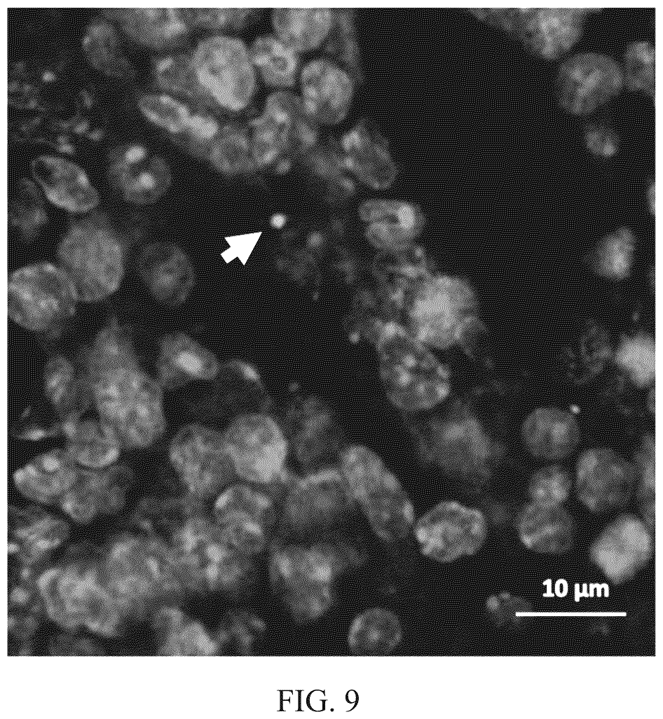

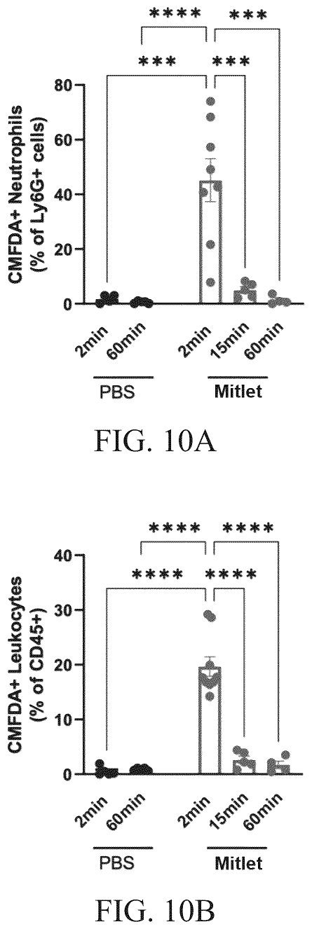

When mitlets were injected into mice in vivo, the mitlets were quickly absorbed by platelets, leukocytes, bone marrow, and speed. As shown in FIG. 9, mitlet uptake by bone marrow tissue in vivo occurs within 15 minutes (as shown) or faster. Other cells that readily absorb mitlets include neutrophils, leukocytes and cells of the bone marrow and spleen. Regarding neutrophils, these cells readily absorb mitlets within 2 minutes of incubation of the former as shown in FIG. 10A. Likewise with leukocytes, mitlets were observed as being absorbed by these important T cells within 2 minutes of incubation with the mitlets. As shown in these data, various cell types hungrily absorb mitlets within mere minutes (or less) of exposure, thereby rapidly increasing not only internal mitochondria count but also mitochondria quality.

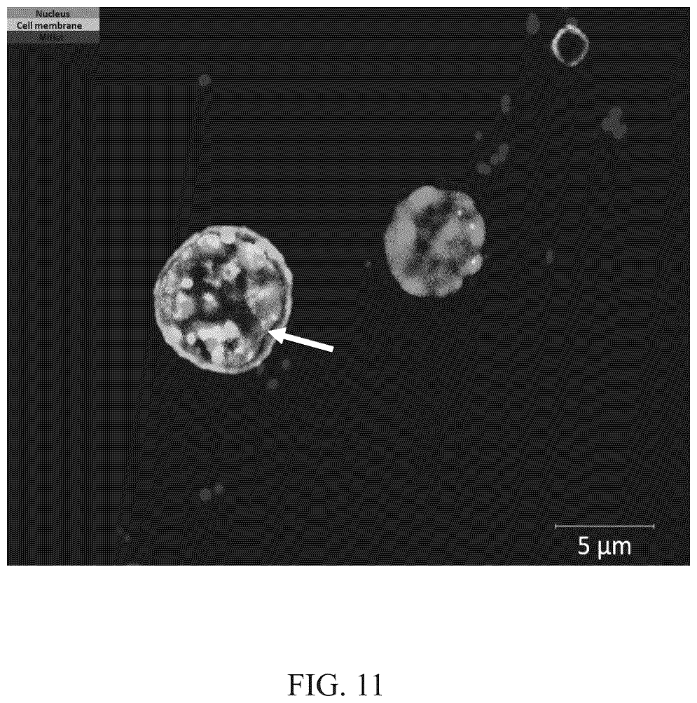

Mitlets were shown to be internalized by T cells in vitro as visualized by the confocal image in FIG. 11. The nuclei of the cells in the image are represented by the blue-labeled cellular structure. The cell membrane in the image is represented by the green-labeled cellular structure; the cell membrane is faintly displayed in the neighboring T cell due to it being slightly outside of the focal plane of the image. Lastly, the mitlets are represented by the red-labeled cellular structure. As the arrow in FIG. 11 indicates, a mitlet is shown to be successfully absorbed by the T cell.

### Example 3

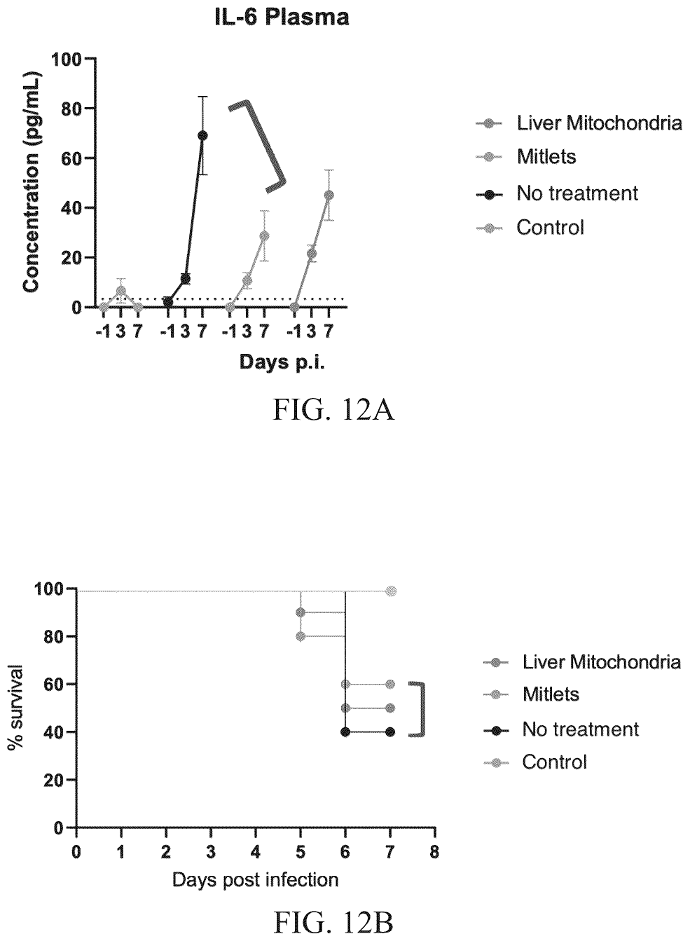

CRS is a major drawback in CAR-T cell therapy, where (in severe cases) CRS manifests with other features of a systemic inflammatory response, including hypotension, hypoxia and/or organ dysfunction—the latter including organs involved in cardiac, pulmonary, hepatic, renal and gastrointestinal systems. Morris E C, et al. “Cytokine release syndrome and associated neurotoxicity in cancer immunotherapy” Nat Rev Immunol. 2022 February; 22(2):85-96. To evaluate the ability of mitlets to mitigate CRS and/or the effects thereof, the murine model of H1N1 infection was used, specifically the H1N1 (PR8) influenza infection model. The groups included in the study were (1) healthy controls without exposure to H1N1, n=5; (2) controls exposed to H1N1 without treatment. (3) those that were administered mitlets, n=10 (5 received mitlets frozen in phosphate buffered saline (PBS) and 5 received mitlets frozen in cryopreservative); and (4) those that were administered fresh liver mitochondria, n=10. Groups (2)-(4) were infected with H1N1. Plasma cytokine levels, specifically interleukin 6 (IL-6) (pg/mL), were measured at different time points: one day prior to infection, and three and seven days post-infection—the results of which shown in FIG. 12A. IL-6 is a pleiotropic, proinflammatory cytokine that is often involved in lethal CRS seen in CAR-T cell treatments, symptoms of which may start manifesting themselves within a few days of T cell infusion. Hirano T, “IL-6 in inflammation, autoimmunity and cancer” Int Immunol. 2021 Mar. 1; 33(3):127-148.

As shown in FIG. 12A, healthy controls that were not exposed to H1N1 show a low baseline plasma concentration of IL-6. However, in the group that was exposed to H1N1 without treatment, levels of IL-6 reached as high as about 70 μg/mL. When treated with frozen mitlets, levels of IL-6 reached only as high about 30 μg/mL, more than 50% less than the group exposed to H1N1 without treatment. Mice administered with fresh liver mitochondria did exhibit a lower level of IL-6 concentration in the plasma. However, mitigation of IL-6 release from mice that received fresh liver mitochondria was not as robust as with the frozen mitlets.

With reference to FIG. 12A, blood was collected from each animal and processed to plasma for cytokine analysis by Luminex (n=5-10 per group). Samples were analyzed in singlicate at a 1 in 4 dilution. Each point represents the group mean±SEM at each time point. The functional Lower Limit of Quantification (LLOQ) (3.34 pg/mL) is plotted as a dotted black line. Values <LLOQ are plotted as 0 pg/ML.

Survival of each group was measured as shown by the Kaplan-Meier survival estimate as shown in FIG. 12B. At day 7, the group that was exposed to H1N1 and had no treatment was the least likely to survival out of all of the groups as shown by its 40% survival rate by day 7. The H1N1 group that received fresh liver mitochondria displayed the next lowest survival rate by day 7. For the H1N1 group that received mitlets that were previously frozen, the survival rate by day 7 was just under 60% yet significantly higher than the H1N1 group without treatment. These results show that mitlet administration may reduce cytokine release (and thus decrease the severity of a cytokine storm) while improving or boosting the immune system in fighting off infections.

### Example 4

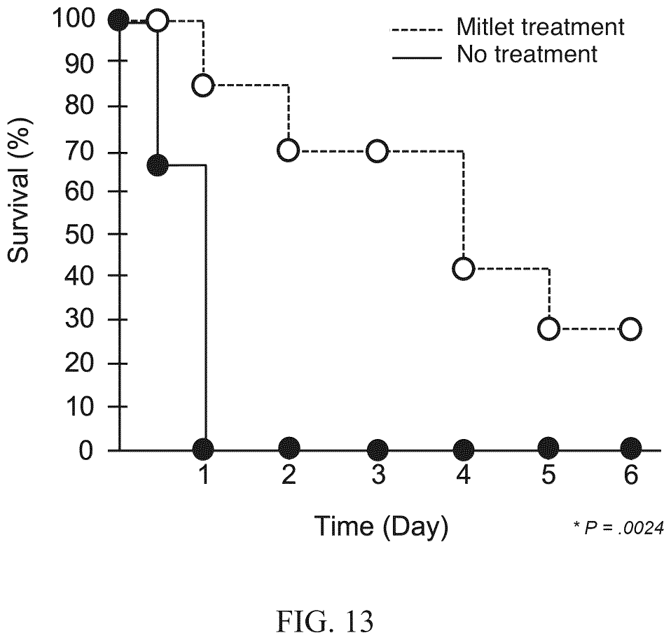

The effect of mitlets on survival and bacterial load in a murine model of sepsis was investigated. The results of which are shown in FIG. 13. The control group included 13-month old mice, n=8. The treatment group included 13-month old mice, n=8, that were treated with fresh mitlets. The mitlets were isolated from 1-month old mice. Essentially, a young source of mitochondria is being administered into middle-aged mice. The treatment schedule included injections of mitlets at 1 day prior to sepsis, and 1, 2, and 3 days after sepsis. Bacterial counts and cytokine levels were collected at 2 days prior to sepsis and 1 and 3 days after sepsis. Survival data was also collected, which is shown in FIG. 13.

The results in FIG. 10 show the mitlet treatment group exhibiting higher rates of survival across every day after inducement of sepsis compared to controls. At one day after inducing sepsis, the mitlet treatment group displayed about an 85% survival rate, whereas all of the controls died by this time. While the survival rate of the mitlet treatment group continued to drop over the 6-day period as shown in FIG. 13, the survival rate of the mitlet treatment group was still significantly higher than the controls. Further, this might hint at a more aggressive treatment of mitlets compared to the treatment schedule used in this example.

### Example 5

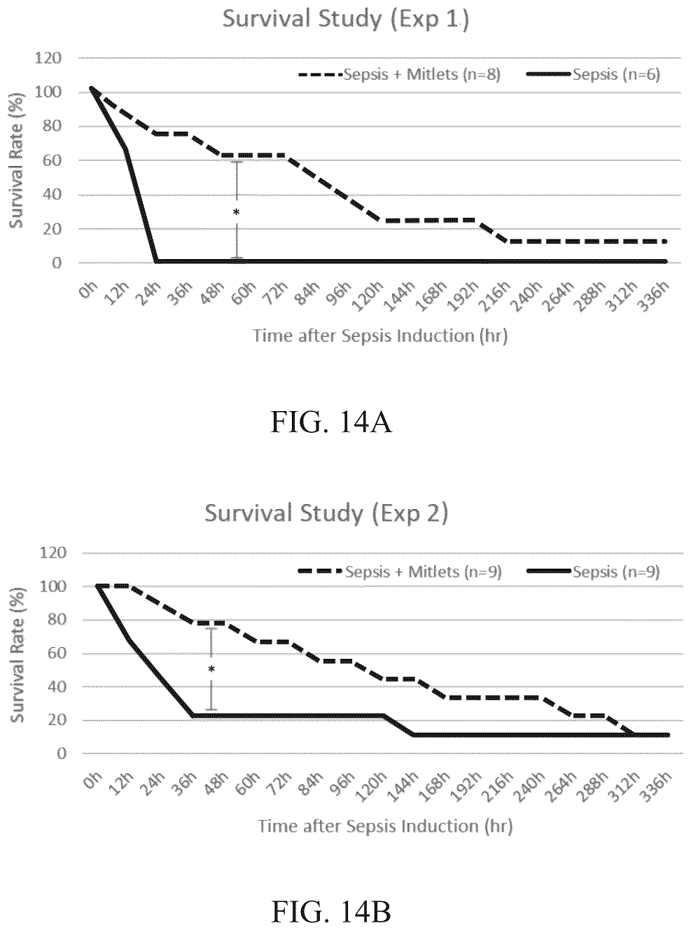

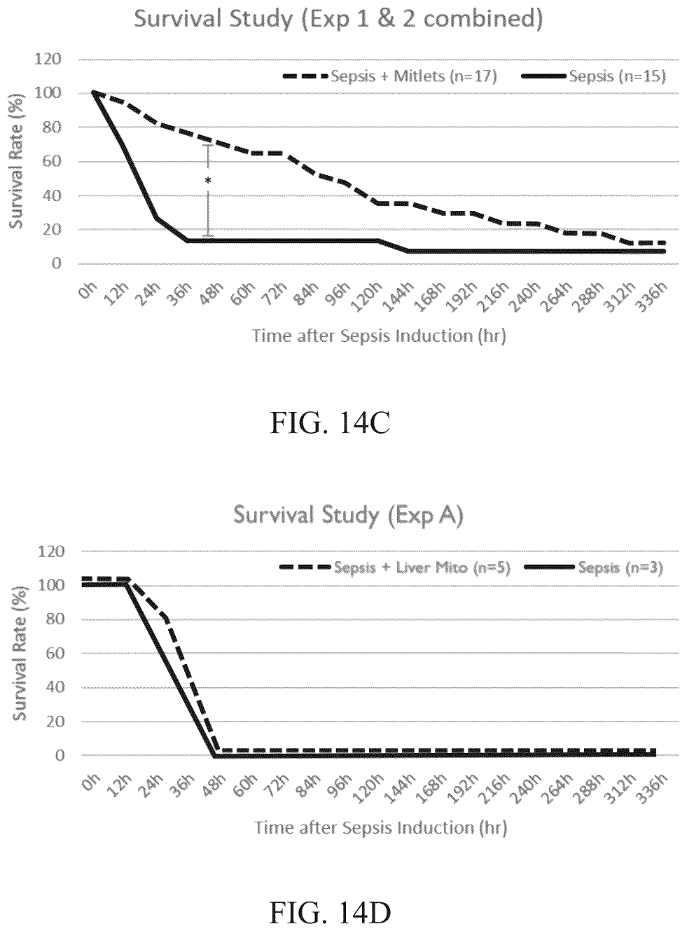

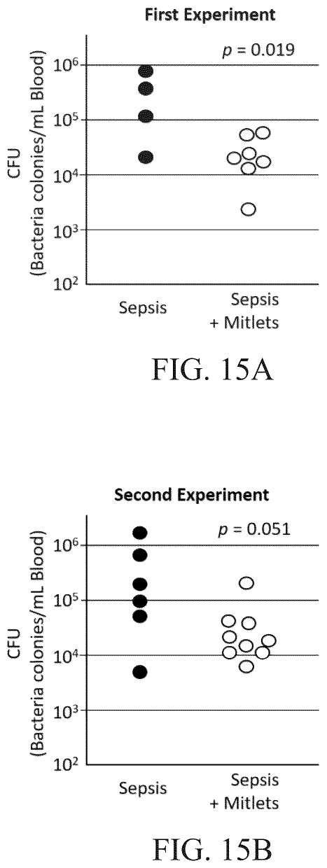

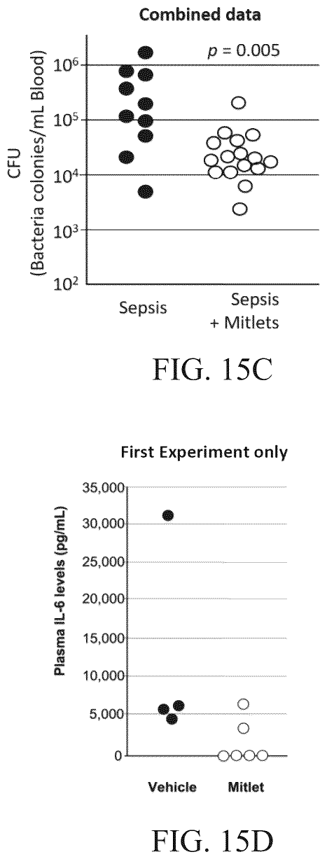

Mitlets isolated from young mice and transplanted into aged recipient mice with sepsis infection significantly improved survival as shown in FIGS. 14A-14C and lowered bacteria counts as shown in FIGS. 15A-15C. Mitlets were isolated from 2-month old mice and administered into 13-month old mice with sepsis. To isolate mitlets, C57BL/6 2-month mouse blood preserved with ACD 20% on ice was purchased. Platelets were isolated from blood, and then washed with Tyrode's Buffer pH 7.4 and resuspended at 1×10⁸ cells/mL in Tyrode's Buffer pH 7.4 with CaCl₂). Platelets were stimulated overnight (16 g) at room temperature with thrombin. To stop the stimulation, 10 mM of EDTA was added and the stimulated platelets were centrifuged at 300 g for 5 minutes to remove remnant platelets or cells. The resulting mix is concentrated by ultracentrifugation at 18,000 g for 60 minutes at 18° C., which isolates approximately 98% of the mitlets according to protein quantification results. The mitlet pellet is resuspended in filtered PBS at pH 7.4. The resulting mitlets were chilled to 4° C. for up to one week prior to administration.

To induce sepsis (polymicrobial abdominal sepsis), bolus injection of cecal slurry (CS) was performed on 13-month old mice. Survival and health of each mouse was monitored multiple times daily for 14 days. Small amounts of blood samples were collected aseptically from the tail vein of each mouse 12 hours after sepsis induction. Each blood sample (10 μL) was diluted with 9-folds volume (90 μL) of 1× citrate solution (0.32% sodium citrate in 0.9% sodium chloride) and plated onto an agar plate. Anaerobic bacteria colonies were counted after the agar plates were incubated for two days.

Isolated liver mitochondria were obtained from 2-month old healthy C57BL/6 mice. Naïve anesthetized mice under isoflurane were euthanized by cervical dislocation, and livers were removed and placed in ice cold homogenization buffer. Livers were transferred to a tissue homogenizer along with 5 ml ice cold homogenizing buffer (300 mM sucrose, 10 mM K-HEPES, and 1 mM K-EGTA (pH 7.2)). The tissue was homogenized for 60 seconds. 250 μL of Subtilisin A (96.61 μM) was added to the homogenate and mixed by inversion. Then, the mixture was incubated on ice for 10 minutes. Using a pre-wet 40 μm filter, the chilled homogenate was filtered into a 50 mL falcon on ice. This filtering step is repeated with another 40 μm filter and then a 10 μm filter. The filtrate was then transferred to a 1.5 mL Eppendorf and centrifuged at 9,000× for 10 minutes at 4° C. The supernatant was discarded, and the pellet was resuspended in 1 mL ice-cold PBS. The concentrated filtrate was then filtered through a 1.2 μm and 0.8 μm filter. A protein assay confirmed the concentration of the isolated liver mitochondria. The concentration of the isolated liver mitochondria was adjusted to 10 mg/kg for administration.

The mitlet administration schedule to the 13-month old mice is summarized in Table 1 below. In the first experiment, mitlets were administered at −1, 1, 2, and 3 days. Bacterial counts shown in FIGS. 15A-15D were determined at −2, 1, and 3 days. In a second experiment, an additional three treatments on Days 3, 4, and 5 were administered.

TABLE 1

| | | Treatments | | | |
|---|---|---|---|---|---|
| Groups | n | Dose | Route | Regimen | Intervention |
| Vehicle control | 8 | N/A | IV, | Day −1, Day 1, | Day 0: |
| with mitlets | | | 120 μL | Day 2, and | Sepsis |
| | | | | Day 3 (x4) | induced |
| Mitlets (in PBS) | 8 | N/A | IV, | Day −1, Day 1, | via Cecal |
| | | | 120 μL | and Day 3 (x3) | Slurry (CS) |
| Vehicle control | 3 | 10 | IV, | Day −1, Day 1, | injection |
| with liver | | mg/kg | 120 μL | and Day 3 (x3) | |
| mitochondria | | | | | |
| Liver | 5 | 10 | IV, | Day −1, Day 1, | |
| mitochondria | | mg/kg | 120 μL | and Day 3 (x3) | |

Referring to FIGS. 14A-14C, mitlet treatments significantly prevented early death one week after sepsis induction (P<0.01). However, mitlet treatments (by Day 2-5) delayed but did not prevent later death. The survival curves of FIGS. 14A-14D were analyzed by Kaplan-Meier LogRank test. Data for two-group comparisons were analyzed by Student t-test. When multiple comparisons were made, the Shapiro-Wilk normality test was run. If the data passed the normality test, one-way ANOVA and Holm-Sidak post-hoc test were used to analyze the data. When the data was not normally distributed, the Kruskal-Wallis test and Dunn post-hoc test were used. Instances were one group was assessed multiple times (i.e., bacterial load), repeated-measures one-way ANOVA was used, and when multiple groups were assessed multiple times (i.e., body temperature data), repeated-measures two-way ANOVA was used and the Holm-Sidak post-hoc test was run. The data shown in FIGS. 14A-14D are expressed as means and standard deviations, where p<0.05 was considered statistically significant.

In contrast to the mitlet-administered groups, those who were administered isolated liver mitochondria showed no protective effect, which is shown in FIG. 14D. The sepsis-induced mice that received the isolated liver mitochondria died at a similar time as negative controls.

Referring to FIGS. 15A-15C, bacterial counts from first and second experiments were lower in mitlet-administered mice compared to the sepsis controls. While the mitlet treatment did not completely eliminate assayed circulating bacteria, the mitlet treatment reduced the bacterial number several fold. As shown in FIG. 15D, mitlet administration reduced IL-6 cytokine levels by about 60%.

### Example 6

To investigate the immunomodulatory function of mitochondrial transfusion, the isolation of immune cells was first to be studied. Two mice were anaesthetized and then sacrificed. Spleen and hind legs were obtained from these mice. Spleens were disrupted to obtain either T cells or NK cells. Bone marrow from one pair of legs (tibia, femur, iliac crest) were flushed to isolate neutrophils. The other pair of legs were flushed to isolate monocytes. Disrupted tissue samples were sorted to recover specific cell types, the results of which are shown in Table 2 below.

TABLE 2

| | Pre-sorting | Post-sorting | | |
|---|---|---|---|---|
| Target cell | Count | Count | Yield | % of Purity |
| Neutrophils | 54.7 million | 9.87 million | 18% | 66.4% |
| Monocytes | 78.2 million | 1.8 million | 2.3% | 84.5% |
| T cells | 96 million | 15.4 million | 16% | 80.5% |
| NK cells | 120 million | 1.63 million | 1.35% | 74.2% |

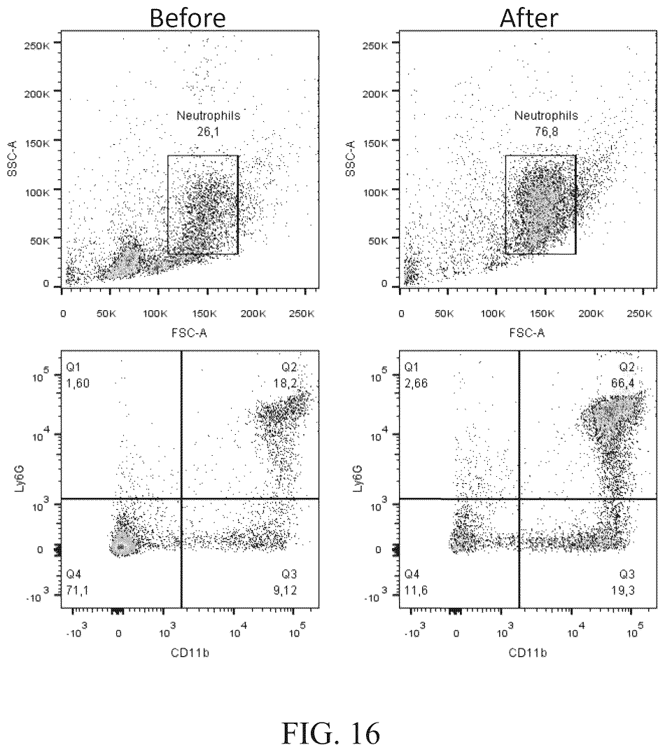

As shown in FIG. 16, after cell-sorting, 66.4% of the sorted cells were CD11b⁺ and Ly6G⁺. CD11b, also known as integrin α_{M}, is a protein subunit that associates with β₂-integrin (CD18) to form compliment receptor 3 (CD3). Also, CD11b is a cell surface antigen expressed by various immunological cells. Ly6G (lymphocyte antigen-6 complex, locus G) is a glycosylphosphatidylinositol (GPI)-linked differentiation antigen that is expressed by myeloid-derived cells. Monocytes express Ly6G transiently during bone marrow development. Granulocytes and neutrophils express Ly6G, which, together with CD11b, are used as markers for these cells. Based on the results shown in FIG. 16, CD11b⁺ neutrophils can be isolated from tissue samples.

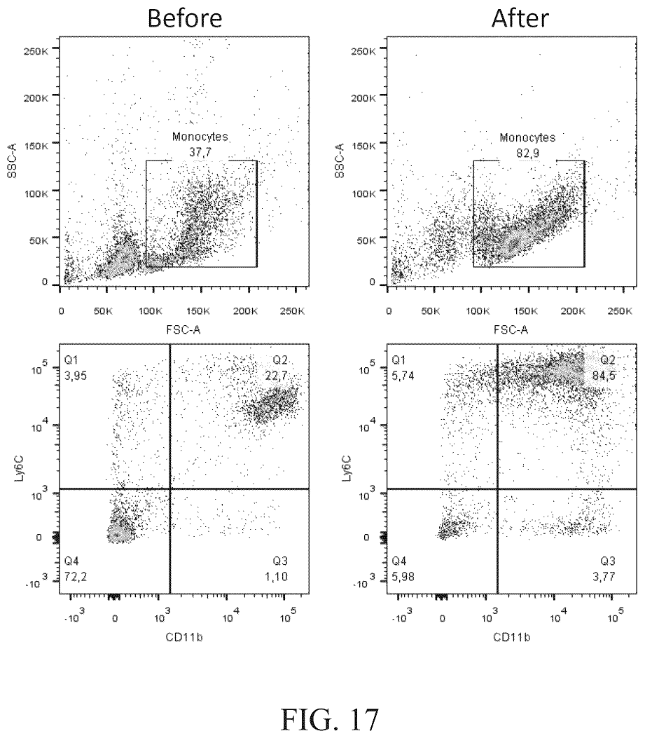

As shown in FIG. 17, after cell-sorting, 84.5% of the sorted cells were CD11b⁺ and Ly6C⁺. Ly6C (lymphocyte antigen-6 complex, locus C) has been used as a surface marker for murine monocytes. Yang P, et al. “Immunological Feature and Transcriptional Signaling of Ly6C Monocyte Subsets From Transcriptome Analysis in Control and Hyperhomocysteinemic Mice.” Front Immunol. 2021 Feb. 25; 12:632333. Based on the results shown in FIG. 17, CD11⁺ monocytes can be isolated from tissue samples.

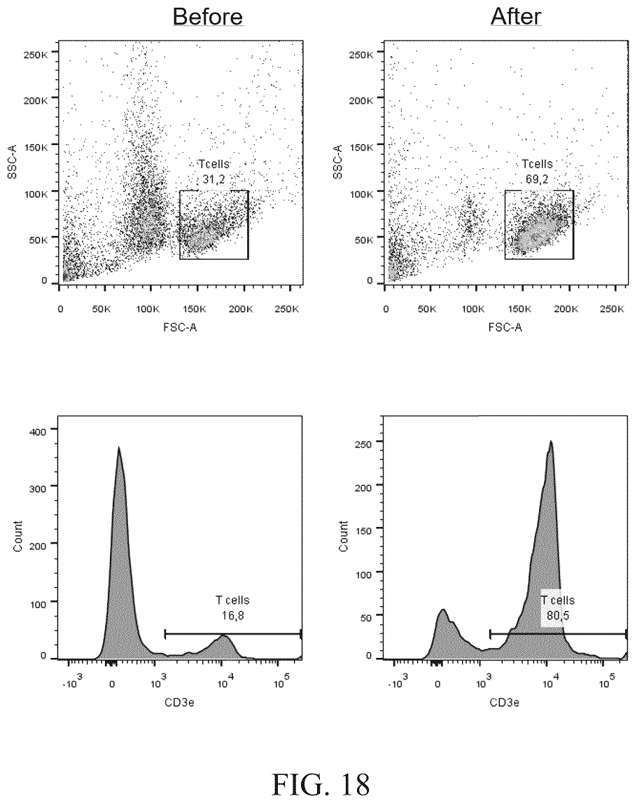

As shown in FIG. 18, after cell-sorting, 80% of the sorted cells are CD3⁺. CD3 is a multimeric protein complex that comprises four distinct polypeptide chains: delta (δ), epsilon (ε), gamma (γ), and zeta (ζ). These chains assemble into three pairs of dimers (e.g., δε, γε, ζζ). CD3 functions as a signal transduction element of T cell receptors and is widely used as a T cell marker. Based on the results shown in FIG. 18, CD3⁺ T cells can be isolated from tissue samples.

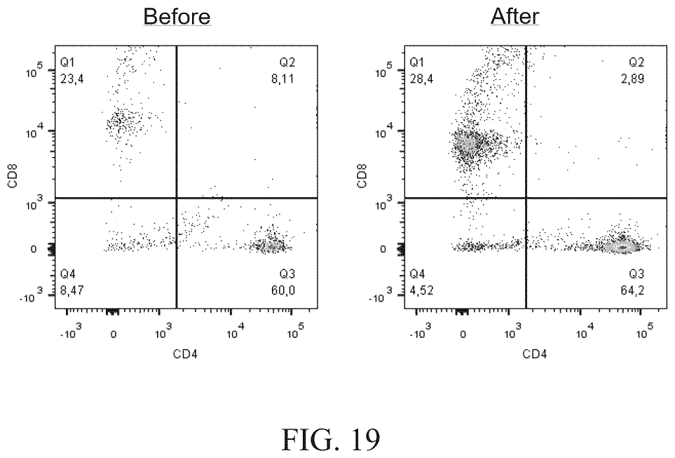

As shown in FIG. 19, after sorting CD3⁺ cells, 64% of the cells were CD4⁺ and 28% were CD8⁺. CD4 is an adhesion molecule that binds to MHC class II molecules and is involved in signal transduction. CD8 is also an adhesion molecule but binds to MHC class I molecules; likewise, CD8 is involved in signal transduction.

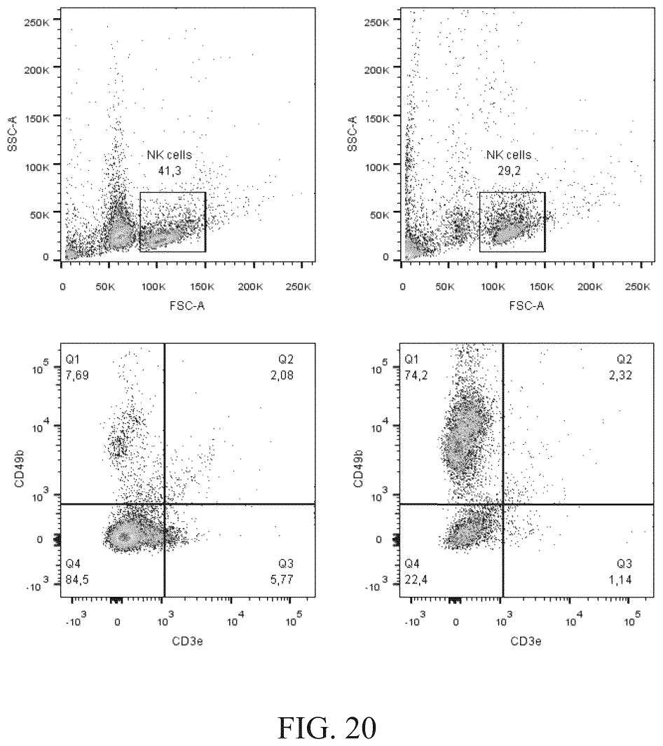

As shown in FIG. 20, after cell-sorting, 74.2% of the sorted cells were CD49b⁺ and CD3⁻. CD49b is also known as integrin α₂, which heterodimerizes with CD29 (integrin β₁) to form very late antigen-2 (VLA-2) complex, receptor for fibrillar collagen and other extracellular matrix (ECM) proteins. Fan X, et al. “CD49b defines functionally mature Treg cells that survey skin and vascular tissues.” J Exp Med. 2018 Nov. 5; 215(11):2796-2814. CD49b is a well-known marker for NK cells. Id. Based on the results shown in FIG. 20, NK cells can be isolated from tissue samples.

### Example 7

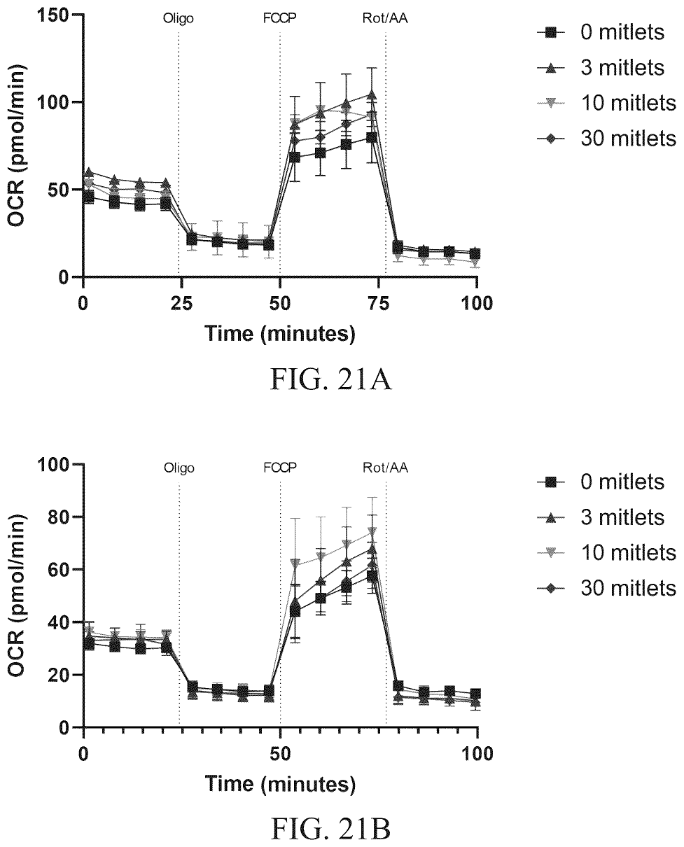

The oxygen consumption rate (OCR) of bone marrow neutrophils (FIG. 21A) and T cells (FIG. 21B) were measured by a Seahorse XF Assay six hours after incubating the cells with mitlets. The Seahorse XF Assay determines the mitochondria stress profile for these respective cell types. During the assay, the OCR was measured between different mitlet incubation levels for bone marrow neutrophils and T cells as shown in FIGS. 21A and 21B respectively. The mitlet levels that were assayed under this assay were 3, 10, or 30 mitlets per cell. Cells in a control group were not incubated with mitlets, and were designated as “0 mitlets” as shown in FIGS. 21A and 21B.

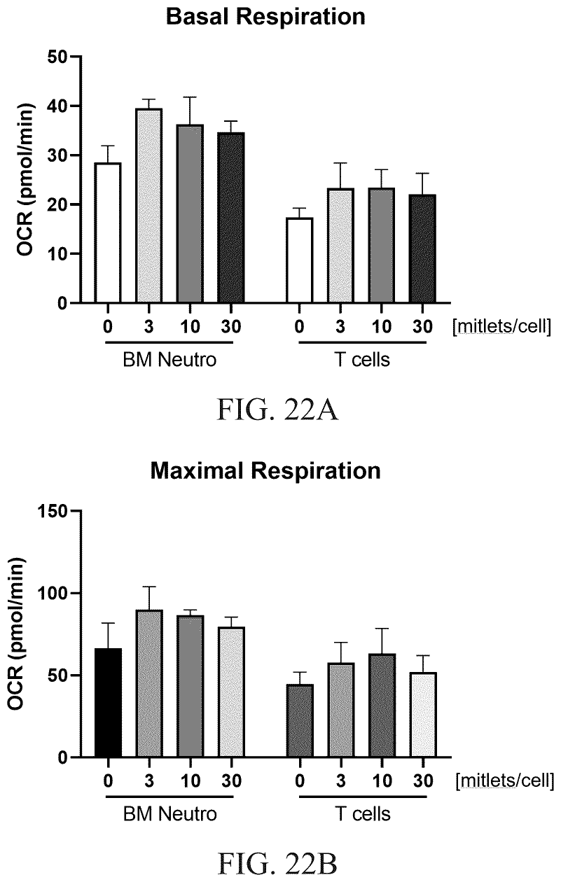

For the Seahorse XF Assay in this example, basal respiration was measured in the first 25 minutes of the assay. Basal respiration measures the oxygen consumption that is used to meet cellular ATP demand resulting from mitochondrial proton leak, which illustrates the energetic demand of the bone marrow neutrophils in FIG. 21A and T cells in FIG. 21B under baseline conditions. As shown in FIG. 21A, the energetic demand of the bone marrow neutrophils is elevated in groups that were incubated with mitlets, particularly the group that received 3 mitlets/cell, which is also represented in FIG. 22A. As shown in FIG. 21B, the energetic demand of T cells also increases following incubation with mitlets with the groups that received 3 and 10 mitlets per cell showing the largest increases over the controls.

The fall in the OCR caused by the injection of the ATP synthase inhibitor oligomycin around 25 minutes as shown in FIG. 21A (neutrophils) and FIG. 21B (T cells) represents the portion of basal respiration that drives ATP production to meet the energetic needs of the respective cells. Moreover, these results are indicative of increased ATP production in neutrophils and T cells that is still occurring 6 hours after absorption of the mitlets. This increased ATP production represents a boost in the energetic capacity of neutrophils and T cells that received mitochondria from the mitlets.

This boost in cellular energetics in neutrophils and T cells is further demonstrated by the maximal respiration reading that reveals the maximal oxygen consumption of the assayed cell following the addition of FCCP (an uncoupler of mitochondrial oxidative phosphorylation) at around 50 minutes as shown in FIGS. 21A and 22B for both cell types. FCCP causes the respiratory chain to operate at maximal capacity by mimicking the physiological energy demand. Adding FCCP causes rapid oxidation of substrates (e.g., sugars, fats, and amino acids) to meet the metabolic demands to achieve the maximum rate of respiration to the respective cells. As shown in FIG. 22B, the maximal respiration is raised in bone marrow neutrophils and T cells that were incubated with mitlets, which demonstrates the effect of the mitochondria payload from the mitlets to enhance or improve cellular energetics of immunological cells, including neutrophils and T cells.

### Example 7

The presence of mitlets with resting (inactivated) and activated T cells to their respective cellular respiration was evaluated. Mitlets were produced from the simulation of the blood from 2 transgenic mice that express fluorescently-labeled markers. The stimulation was performed with thrombin (0.5 U/ml) overnight and the mitlets were then concentrated by ultracentrifugation. T cells were extracted from the spleen of a WT mouse. Here, a higher number of T cells were obtained than usual. A subset of the extracted T cells were activated overnight with αCD3/αCD28 beads in the presence of mitlets at 0, 3 or 30 mitlets/cell. Additionally, naked mitochondria (“mitos”) was also tested with activated T cells. Mitochondrial respiration was tested using the Seahorse XF assay, which measures the OCR for various parameters involved in cellular respiration. Intake was measured by flow cytometry and microscopy.

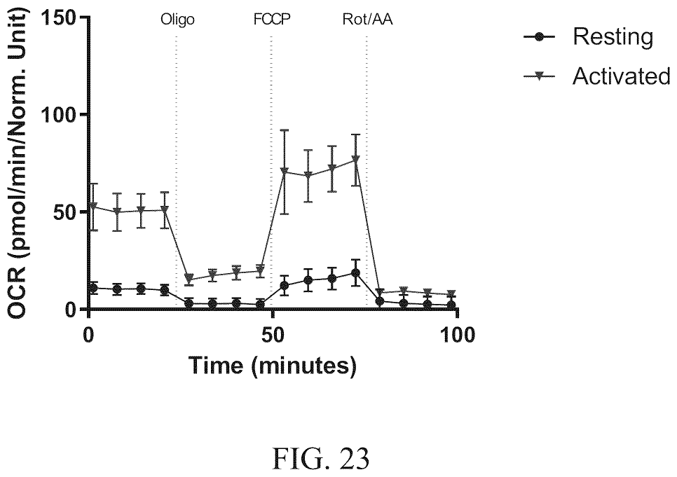

As shown from the results of the Seahorse assay in FIG. 23, resting mitochondria and activated mitochondria (both groups without mitlets or mitos) expectedly showed different levels of oxygen consumption. The activated T cells, as expected, showed a significantly increased OCR after 24 hours, especially with respect to the basal respiration (prior to oligo injection), ATP production (between oligo and FCCP injection), and maximal respiration (between FCCP and rotenone and antimycin A injections) readings from the assay.

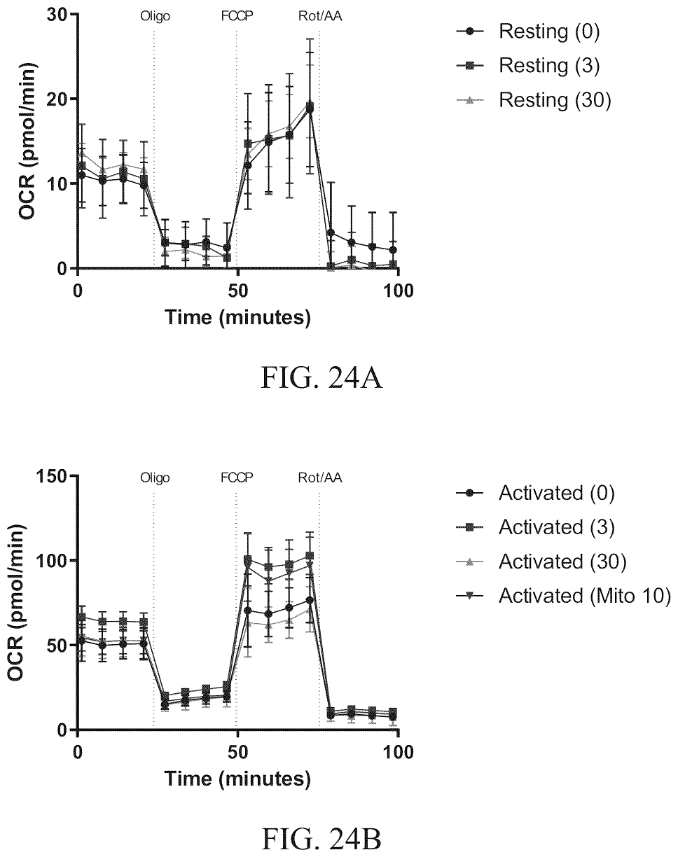

FIGS. 24A and 24B show the cellular respiration of resting and activated T cells respectively in the presence of mitlets after 24 hours. FIG. 24A shows how resting T cells in the presence of mitlets after 24 hours display little difference other than a small increase in the basal respiration when compared to the control (0 mitlets/cell). FIG. 24B shows how activated T cells exhibited an increase in basal respiration in the presence of mitlets as low as 3 mitlets/cell. Additionally, there is a trend towards increased maximal respiration with mitlets as low as 3 mitlets/cell with the activated T cells. Additionally, activated T cells in the presence of mitos (10 mitos/cell) did not display an increase in basal respiration as seen with the mitlets (3 mitlets/cell).

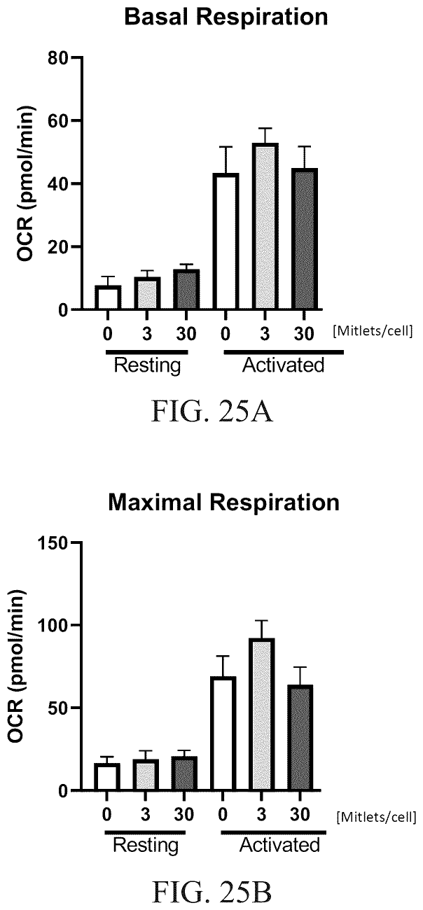

FIGS. 25A and 25B show the basal and maximal respirations of the resting and activated T cells in the presence of mitlets after 24 hours. For basal respiration shown in FIG. 25A, a small increase in the respiration of resting T cells was observed, whereas the increase in the basal respiration of the activated T cells in the presence of mitlets (3 mitlets/cell) was much larger. For maximal respiration shown in FIG. 25B, an increase was only observed in activated T cells in the presence of mitlets (3 mitlets/cell).

These results indicate that activated T cells readily absorb mitlets, whereas resting T cells appear to be less inclined to do so. This absorption is a controlled action that depends on T cells being in an activated state. Further, this absorption activity is basically the same as observed in neutrophils and other immunological cell types. These findings indicate that mitlets are a form of a “battery park” that is interchangeable between all types of immune cells.

### Example 8

To obtain mitlets including mitochondria and a coating applied to the mitochondria, the coating process is performed after expansion of the mitochondria. The coating process includes preparation of the AsOR. To prepare AsOR, 10 mg of α-acid glycoprotein (orosomucoid, OR from human plasma, Sigma Cat #G9885) is dissolved in 5 ml MilliQ water. Afterwards, 5.0 ml of 0.2 N H₂SO₄ is added as an equal volume. This mixture is heated at 80° C. for 1 hour in a water bath. Dialyze the heated mixture through a pre-soaked dialysis membrane (10 kDa MW cut-off) with 10 changes of 1 L sterilized MilliQ water for over 48 hours at 4° C. The dialyzed protein is then stored frozen in 1 ml aliquots or lyophilized.

AsOR-Dylight is prepared by adding 200 μg (200 μl of 1 M NaHCO₃, Alfa Aesar, Cat. #14707) to 1 mg (2 ml of AsOR, 0.5 mg/ml). After, 50 μg of Dylight 488 NHS ester (ThermoFisher, Cat. #46403) freshly dissolved in 50 μl of DMF (Sigma-Aldrich, Cat #227056-1L) is immediately then added to the AsOR solution, capped and placed on rocker platform (gentle shaking) for 1 hour at room temperature. Meanwhile, 1 L MilliQ water at 4° C. is pre-chilled. After the shaking is finished, the reaction mix is then put in pre-soaked dialysis tubing (ThermoFisher, Cat #88243, 10 kD MW cutoff membranes), and dialyzed against 3 changes 1 L of MilliQ water over 24 hr at 4° C. If subsequent conjugation reactions are to be done, sample is dialyzed twice against 150 ml of 0.1 M MES pH 6.0 (prepared from 1 M stock, Alfa Aesar, Cat #J61656) for buffer exchange.

To prepare AsOR-PL, 500 μg of poly-L-lysine (PL) (50 μl of 1 mg/100 μl in 0.1 M MES pH 6 (Sigma-Aldrich, Cat #P0879) is added to 1 mg AsOR-Dylight in approximately 2.0 ml MES pH 6 and mixed. 500 μg of fresh EDC (50 μl of 1 mg/100 μl EDC stock in 0.1 M MES pH 6) is added to AsOR-Dylight and PL mixture and incubated on rocker platform (gentle shaking) for 1 hour at room temperature. The reaction mix is concentrated, and buffer exchanged using a spin filter having a 10 kD MW cut-off (Pierce catalogue #88513, 0.5 ml capacity) according to vender's protocol using 8 volumes of 0.5 ml 0.1 M MES pH 6.0. Check filtrate until A²³⁰ is the same as background. Sterile filter (0.45μ) the filtrate and store at 4° C. or lyophilize.

To prepare AsOR-LLO, 1 mg of AsOR in 200 μl of PBS, pH 7.4+150 mM NaCl+1 mM EDTA is reacted with 1 mg of SPDP (freshly prepared in 50 μl of DMSO) and brought to a final volume of 2000 μl with 1×PBS, pH 7.4+150 mM NaCl+1 mM EDTA. Afterwards, this reaction mix is incubated for 1 hour. After incubation, the reaction is concentrated and washed to remove free SPDP using a spin filter (10 kDa MW cut-off, 0.5 ml capacity) by washing 8 times with 0.5 ml PBS, pH 7.4+150 mM NaCl+1 mM EDTA. The filtrate is checked under A²⁶⁰ (for SPDP) is baseline.

240 μg of purified His-LLO (240 μg/1 ml in PBS, pH 7.4+150 mM NaCl+1 mM EDTA) is treated with 0.5 ml of 150 mM DTT (freshly prepared in PBS, pH 7.4+150 mM NaCl+1 mM EDTA). Final concentration of DTT 50 mM. This reaction is incubated at room temperature with gentle shaking for 1 hour. After 1 hour of incubation, the DTT-treated His-LLO is concentrated and washed 4 times with 0.5 ml PBS, pH 7.4+150 mM NaCl+1 mM EDTA and a spin filter having a 10 kD MW cut-off to obtain a final volume of 130 μl. 240 μg in of His-LLO is passed through buffer saturated 0.5 ml Zeba Spin molecular sieve columns with 7 kDa MW cutoff to remove any remaining traces of DTT. The final 240 μg His-LLO in 130 μl is reacted with 360 μg AsOR-SPDP in a final volume of 1 ml PBS, pH 7.4+150 mM NaCl+1 mM EDTA. Final molar ratio of LLO:AsOR is 1:1.5. The reaction mix is then placed on a platform rocker set to low speed in the cold room at 4° C. for overnight incubation.

After overnight incubation, an aliquot of the sample was removed. A²⁸⁰ nm absorbance to measure PDP bound to protein, and A³⁴³ nm absorbance to measure 2-pyridine dione released by addition of DTT. Buffer is alone or with DTT as blank, respectively. Additionally, after overnight incubation, the reaction mix is purified as described below or put into pre-soaked dialysis tubing 10 kD MW cutoff and dialyzed 3 times against 20 L MilliQ water over 24 hrs, and sterile filtered for storage at 4° C.

To prepare the histidine tag-LLO (His-LLO), bacterial stock (Plasmid number #DP-E3570 strain) is streaked onto LB/Km 100 μg/ml agar plate. The plate is incubated at 37° C. for 24 hours. A single colony from the plate is inoculated in 25 ml LB/Km (100 μg/ml working concentration) broth in 125 ml culture flask and placed on an orbital shaker at 225 rpm for 48 hours at 37° C. The culture is diluted 1:100 times in fresh LB/km (50 μg/ml working concentration) broth 1000 ml/2 L flask. The flask is incubated with shaking at 225 rpm at 37° C. for 3 hours. After 3 hours of growth, 0.5 final concentration of IPTG is added to the bacterial culture. The culture is allowed to grow for 5 hours more before harvesting the cells by centrifugation at 8500 rpm (6,540×g) at 4° C. for 5 minutes. The bacterial cells are washed once by resuspending the cell pellet in 40 ml of cold 1×PBS, pH 7.4, and cell suspension is centrifuged at 8500 rpm (6,540×g) at 4° C., for 5 minutes, and the clear supernatant is carefully discarded in a flask containing commercial bleach. The cell pellet is stored at −80° C. until used for protein extraction.

For protein extraction, the bacterial cell pellet is resuspended in 5 ml lysis buffer (50 mM sodium phosphate buffer pH 8 with 1 M sodium chloride, 10 mM 2-mercaptocthanol, 20 mM imidazole and 1 mM PMSF). Cells on ice are lysed by mechanical disruption using MP-Biomedical FastPrep-24 classic system with 40 seconds pulse at a speed setting of 6.0 m/s (6 pulses with one-minute intervals after each pulse). Cell lysate is spun at 8500 rpm (6,540×g) for 10 min at 4° C. The clear lysate is then passed twice through a cobalt agarose-column pre-equilibrated with lysis buffer. The final flow-through was collected and saved. The column is washed 4 times with 3 ml of lysis buffer, and twice with wash buffer (50 mM sodium phosphate buffer pH 6 with 1 M sodium chloride, 10 mM 2-mercaptocthanol, 20 mM imidazole, 1 mM PMSF, 5% glycerol, 0.1% Tween 20). His-LLO is eluted with 3 ml of elution buffer (50 mM sodium phosphate buffer pH 6 with 1 M sodium chloride, 10 mM 2-mercaptoethanol, 800 mM imidazole, 1 mM PMSF). Elution is continued until A²⁸⁰ of eluate is baseline. His-LLO is concentrated and buffer exchanged with storage buffer (50 mM sodium phosphate buffer pH 6 with 1 M sodium chloride, 10 mM 2-mercaptocthanol, 20 mM imidazole, 1 mM PMSF and 1 mM EDTA), using spin filter Pierce 30 kDa MW cut-off. The protein concentration was estimated using a NanoDrop UV-Vis Spectrometer by measuring A 280.

To prepare and purify poly-L-lysine-Dylight-LLO, first PL 1 mg (5-10 kD Sigma-Aldrich) in 1.0 ml (PBS pH 7.4+150 mM NaCl+1 mM EDTA) to which is added 0.4 mg of SPDP (dissolved in 40 μl of DMSO) to make the final reaction volume to 1.0 ml with the buffer. Then, incubate the reaction at room temperature for 1 hr. After incubation, concentrate and wash the sample with 3 to 4 times with 0.5 ml of the buffer (PBS, pH7.4+150 mM NaCl+1 mM EDTA) using a spin filter Pierce 10 kD MW cut-off (Thermoscientific Cat. #88513) to concentrate the sample to 130 μl and pass it through one 0.5 ml Zeba Spin molecular sieve columns, 7 kD MW cutoff (Thermoscientific Cat #89882) to remove any remaining unreacted SPDP. PL-SPDP 0.5 mg in 1.0 ml PBS [pH 7.4+150 mM NaCl+1 mM EDTA] is reacted with 50 mg of Dylight 488 (dissolved in 50 μl of DMF) and make the final reaction volume to 1.0 ml. Incubate the reaction volume at room temperature for 1.0 hr.

After incubation, concentrate and wash sample 4 times with 0.5 ml PBS, pH 7.4+150 mM NaCl+1 mM EDTA using the 0.5 ml spin filter with 10 kD MW cut-off. Pass sample through two 0.5 ml Zeba molecular sieve spin columns 7 kD MW cut-off to concentrate the sample to 130 μl. His-LLO (cobalt-agarose-column purified) 0.38 mg is mixed with 11.5 mg dithiothreitol (DTT), 0.5 ml of 23 mg/ml dissolved in PBS pH7.4. The final reaction volume was brought up to 1 ml with PBS pH7.4 and incubated at room temperature for 1.0 h.

After incubation, concentrate and wash sample 4 times with 0.5 ml PBS, pH 7.4+150 mM NaCl+1 mM EDTA using a 0.5 ml spin filter with 10 kD MW cut-off. Concentrate the retentate to 130 μl and pass it through two 0.5 ml Zeba molecular sieve spin columns 7 kD MW cut-off to eliminate DTT. Activated His-LLO 0.38 mg was reacted with PL-Dylight-SPDP 0.5 mg in 130 μl in a molar ratio of LLO:PL of 1:3 in a final reaction volume of 1 ml with buffer pH 7.4 (PBS+150 mM NaCl+1 mM EDTA). Incubate the reaction overnight at 4° C. with gentle shaking. Into 900 μl of reaction mix, add 900 μl of 2× binding buffer A pH8 (100 mM sodium phosphate+2.0 M sodium chloride) and mixed. Load the reaction mixture on a 5 ml column (Qiagen, Cat #34964) containing 2.5 ml Ni-NTA-agarose (Qiagen, Cat #30210), pre-saturated with binding buffer B pH 8 (50 mM sodium phosphate, 1.0 M sodium chloride, 20 mM imidazole and 1.0 mM PMSF). Collect the flow-through and wash column washed once more with the same buffer and collect. Wash the column with 3 times with 3 ml of binding buffer B. Wash twice with 3 ml buffer (50 mM sodium phosphate, pH 6, 1.0 M sodium chloride, 20 mM imidazole, 1.0 mM PMSF, 5% glycerol and 0.1% Tween 20).

Finally elute the HIS-LLO with 3 ml elution buffer, pH 6 (50 mM sodium phosphate, 1.0 M sodium chloride, 800 mM imidazole, 1.0 mM PMSF, 1 mM EDTA). Repeat elution with 1 ml of elution buffer, and pool the eluates to yield 4 ml of eluate. Spin filter eluate, 4 ml, through Pierce spin filters 10 kD MW cut-off 0.5 ml capacity pre-equilibrated with storage/shipping buffer, pH 6 (50 mM NaH2PO4, 1 mM EDTA, 2.7 mM KCl, 5% (v/v) glycerol and 0.5 M NaCl) collecting the total retentate in 400 μl eliminating free PL-SPDP. If necessary, to eliminate high molecular weight products, pass retentate through a spin filter Pierce 100 kD MW cut-off 0.5 ml capacity collecting the retentate. The sample can be stored at −20° C. until used for further analysis.

To titrate the AsOR-Dylight-PL (AsOR-PL) conjugate with mitochondria (e.g., bioreactor-grown mitochondria), 128 μg mitochondria, are used fresh or thawed on ice, pooled in one tube and washed twice with 1 ml of PBS, pH 7.4 by spinning at 12,000 ref for 10 min at 4° C. The final pellet is resuspended in 250 μl of PBS. The protein concentration is measured using bicinchoninic acid (BCA) or Bradford assay. Increasing amounts (0.25 to 2.0 μg) of AsOR-PL are added to constant amounts 128 μg of mitochondria, and addition of PBS makes a final volume of 50 μl. These complexes are incubated on ice for 45 minutes to allow the mitochondria to bind AsOR-PL, thereby coating the mitochondria with AsOR-PL to obtain the mitlets. After incubation, mitlets are separated by centrifugation at 12,000 ref for 10 min at 4° C. The supernatants are removed, and mitlet pellets are resuspended in 50 μl of fresh PBS. Pellets and supernatants are applied separately on agarose gels or HPAGE with controls.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the phrases “cells grown in culture” or “a tissue grown in culture” refers to a multitude of cells or a tissue, respectively, grown in a liquid, semi-solid or solid medium, outside of the organism from which the cells or tissue derive. In some embodiments, cells grown in culture are cells grown in bioreactors. According to a non-limiting example, cells may be grown in a bioreactor, followed by isolation of mitlets from the cells.

As used herein “therapeutically effective amount,” “therapeutic amount,” or the like refers to an amount of the compound, composition, or mixture that will elicit a desired biological or medical response of a tissue, system, animal, or human, e.g., the response is to a disease or disorder and the amount results a response that reflects at least a partial amelioration of the disease or disorder, or symptoms associated with the disease or disorder.

As used herein, “administrating” and “administration of” are terms that refer to the providing of a composition in a therapeutically effective amount to a subject (including patients) in need of treatment. The routes of administration can be topical, enteral, or parental.

As used herein “mitlets” include mitochondria that are either encased in vesicles or coated. In some examples, the mitlets include a vesicle and mitochondria encased in the vesicle. In some examples, the mitlets (including the vesicle and mitochondria) are emitted or expelled by platelets. In some examples, mitlets are derived from various sources, grown and expanded in bioreactors. In some examples, the vesicles include receptors that target certain cell types. In some examples, the certain cell types include immunological cells. In some examples, mitlets include mitochondria that are coated. In some examples, the coating includes an asialoglycoprotein (AsG). In some examples, the AsG includes asialoorosomucoid (AsOR). In some examples, the coating further includes poly-L-lysine that is linked to the AsOR. In some examples, the coated mitochondria are complexed to a conjugate. In some examples, the conjugate includes AsOR. In some embodiments, the conjugate further includes listeriolysin O (LLO).

As used herein, the term “bioreactor” includes an apparatus or device constructed to support the growth and proliferation of biological entities under controlled and regulated environmental conditions. The bioreactor includes a containment chamber, which is constructed from biocompatible materials to minimize any negative interaction with the biological entities it houses. This chamber provides a secluded and controlled environment, effectively preventing contamination from external sources and promoting optimal growth conditions for the biological entities to grow or proliferate therein in the presence of appropriate media. The “bioreactor” may further include a series of sensors designed to continuously monitor and record critical parameters including, but not limited to, temperature, pH, oxygen and carbon dioxide concentrations, and nutrient and waste product levels. The feedback from these sensors is essential to inform adjustments in the bioreactor's internal environment, thereby maintaining optimal growth conditions. The bioreactor may further include a control system operatively connected to these sensors and to the containment chamber. This control system interprets data from the sensors, and in response, adjusts the environmental conditions within the containment chamber. The controlled manipulation of these parameters allows for the fine-tuning of the biological entity's environment, leading to enhanced growth and productivity. The “bioreactor” may be configured as an input/output system structured for the introduction of fresh media, necessary for providing nutrients, and the removal of spent media, crucial for the elimination of waste products. This system ensures a dynamic environment within the containment chamber, supporting the sustained viability and optimal productivity of the biological entities.

### ADDITIONAL NOTES

The section headings used herein are for organizational purposes only and are not to be construed as limiting the subject matter described.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise. While several examples have been described in detail, it is to be understood that the disclosed examples may be modified. Therefore, the foregoing description is to be considered non-limiting.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of treating a subject with chimeric antigen receptor (CAR)-containing cells, wherein the subject has an indication that is treatable by the CAR-containing cells, the method comprising:
obtaining mitlets as mitochondria derived from platelets;
co-incubating the mitlets with the CAR-containing cells, such that the CAR-containing cells uptake an amount of the mitlets into the CAR-containing cells effective to enhance metabolic activity of the CAR-containing cells; and
delivering an amount of the CAR-containing cells with enhanced metabolic activity to the subject.
